(19) United States
(12) Patent Application Publication
(10) Pub. No.: US 2017/0198337 A1
(43) Pub. Date: Jul. 13, 2017

(54) HIGH THROUGHPUT TELOMERIC CIRCLE ASSAY

(71) Applicant: SENS FOUNDATION, INC., Mountain View, CA (US)

(72) Inventors: Haroldo Souza SILVA, Richmond, CA (US); David Henry HALVORSEN, San Jose, CA (US)

(21) Appl. No.: 15/312,885

(22) PCT Filed: May 20, 2015
(86) PCT No.: PCT/US2015/031831

§ 371 Date: Nov. 21, 2016

Related U.S. Application Data
(60) Provisional application No. 62/001,036, filed on May 20, 2014.

(51) Int. Cl. C12Q 1/68

(52) U.S. Cl. CPC C12Q 1/6823

(57) ABSTRACT

The invention relates to methods and assays for high-throughput, rapid, and quantitative detection of Alternative Lengthening of Telomeres (ALT) activity in cells. The methods and assays involve detecting or assaying for partially double-stranded nucleic acid in a high-throughput format amenable to high-throughput screening optionally utilizing automation, wherein the presence of said circles is specific for cells comprising an active ALT mechanism. In some embodiments the methods find application in, inter alia, determining the level of ALT activity in a cell, determining the ALT status of a cancer in a subject, diagnosing and/or treating disease, determining disease status, analysis of treatment efficacy, and the identification of novel therapeutic agents in large-scale formats.

### I. RELATED APPLICATION DISCLOSURE

This application claims the benefit of U.S. Provisional Application Ser. No. 62/001,036, filed Apr. 20, 2014, which is hereby incorporated in its entirety.

### II. SEQUENCE LISTING DISCLOSURE

This application includes as part of its disclosure a biological sequence listing in the file named “4873101213.txt” created in May 20, 2015 and having a size of 345 bytes, which is hereby incorporated by reference in its entirety.

### III. FIELD

The present disclosure generally relates to the field of nucleic acid detection, particularly in a high-throughput setting.

### IV. INTRODUCTION

Somatic cells have a limited capacity to grow and generate progeny. The role of telomeres in regulating cell division, in particular the number of cell divisions a cell lineage can undergo, is quintessential for this process. Telomeres are repetitive DNA sequences, typically G-rich on one strand (C-rich on the complementary strand), at the termini of linear chromosomes. In human (and indeed most vertebrate) chromosomes telomeres comprise typically several thousand copies of the sequence 5′-TTAGGG-3′ with a G-rich overhang on the 3′ end. Typically, telomeres shorten with each round of cell division, at least in part due to the incomplete replication of the ends of linear chromosomes. When telomeres become too short this evokes normal cellular DNA damage repair pathways. A complex series of biochemical and morphological changes ensues resulting in cell cycle arrest and cell death, either via replicative senescence or via programmed cell death such as apoptosis. Senescence and apoptosis each constitute major pathways for the regulation of cell proliferation. These processes are beneficial, for example, in the suppression of tumorigenesis and limiting disease progression more generally. In pathological conditions characterized by aberrant cellular proliferation, such as cancer, the normal senescence and apoptotic pathways are circumvented enabling cells to become immortal.

To escape from the normal limits on proliferative potential, cancer cells frequently employ a means to counteract the gradual telomere attrition that accompanies DNA replication. While most cancers rely on telomerase for immortalization, a significant proportion of tumors show no detectable telomerase activity. These cancers often use a homologous recombination-based pathway known as alternative lengthening of telomeres (ALT). Unlike the telomerase-based pathway, no ALT-specific targets or therapeutics are yet available due in part to a lack of robust assays to quantitatively measure the ALT-associated biomarkers identified to date.

Cells of a number of human tumors utilize ALT, especially those arising in brain, bone and connective tissue, but the full extent of the role and importance of ALT in many cancers is yet to be fully elucidated. The prognosis for patients with an ALT[+] cancer is generally poor, with median survival ranging from 2 to 5 years. While the presence of any telomere maintenance mechanism in tumors is often associated with poor prognosis and survival, patients diagnosed with liposarcomas and osteosarcomas that utilize the ALT pathway die significantly faster than patients with telomerase-positive tumors.

Both telomerase and ALT represent attractive targets for anti-cancer treatment and there is an increasing recognition that for many cancers it will be desirable to have available therapies that are specific for ALT[+] cells and therapies that are specific for telomerase[+] cells. Cancer cells of a specific cancer type in one individual may utilize telomerase for telomere maintenance, whereas cells of the same cancer type in another individual may utilize ALT, and indeed cells within a single tumor may utilize different telomere maintenance mechanisms (i.e., tumor mosaicism). It is also thought that cancer cells may have the capacity under certain conditions to switch between telomerase-induced telomere maintenance and ALT, raising the possibility that as telomerase-specific cancer therapies increase in clinical use, the prevalence of ALT[+] cancer cells may increase. Thus, with the development of ALT-specific and telomerase-specific therapies, it may be beneficial to determine whether the cancer in any given individual is ALT[+] or telomerase[+].

In those cases where telomerase cannot account for increases in telomere length, recombination-mediated ALT pathway(s) have been proposed, which can augment telomere length by thousands of base-pairs within a few cell cycles. The precise mechanisms involved in ALT activity are not fully understood. However, the ALT mechanism is known to cause abnormal telomere metabolism that generates linear and circular extra-chromosomal telomeric repeats, including partially double-stranded C-rich (CCCTAA)ₙ circles or C-circles (CC). ALT activity is strongly correlated with and proportional to CC levels.

The present disclosure provides fast, high-throughput method for detecting C-circles, which can be used to identify ALT positive cancer cells and tumors and to assess the level of ALT activity within a cell. In exemplary embodiments, assays described herein utilize fluorescence- and ELISA-based methods to quantitatively measure C-circles in human ALT cells. These methods can be effectuated in a high-throughput format, for example utilizing multiwell plates. Working examples herein validate these methods using human cell lines known to depend on ALT activity. Complete results were obtained in as little as one hour after rolling circle amplification (RCA), or within 8 hours, including genomic DNA (gDNA) isolation. The method can be performed by detecting the signal with samples in solution throughout the entire assay, without any need for blotting DNA onto membranes, which allows the assay to be performed by automated liquid handlers. These methods exhibited a broader linear response compared to other methods, without loss of sensitivity. The described methods can also be used to measure alteration (increases or decreases) in ALT activity, such as in a high-throughput screening assay. Agents that decrease ALT activity may be used as potential cancer therapeutics or as lead compounds in the development of same. Conversely, detection of increased ALT by an agent could indicate that the agent may pose a risk of promoting cancer development if given to a subject. Identified agents that increase ALT could also be utilized in research, for example to promote development of ALT-mediated cellular immortality in a controlled fashion.

### V. SUMMARY

One aspect of the invention provides methods for detecting circular nucleic acid, said method comprising the steps of (a) providing a sample comprising a partially double-stranded nucleic acid, said nucleic acid comprising a circular template strand and a linear primer strand; (b) extending the linear primer strand to generate a rolling circle amplification product; and (c) detecting the rolling circle amplification product, wherein detecting the rolling circle amplification product comprises (1) hybridizing a detectable nucleic acid probe to the rolling circle amplification product to form a double-stranded complex, wherein the detectable nucleic acid probe comprises a donor fluorophore and an acceptor fluorophore attached to the detectable nucleic acid probe at positions separated by at least one labile bond; (2) cleaving the labile bond of detectable probe hybridized to the rolling circle amplification product with a nuclease specific for double-stranded nucleic acids; and (3) detecting fluorescence of the donor fluorophore separated from the acceptor fluorophore. In some embodiments, the step of extending the primer strand comprises: (1) providing DNA polymerase and nucleoside triphosphates (dNTPs); and (2) incubating the sample, DNA polymerase and dNTPs under conditions suitable to extend the linear primer and synthesize a single linear strand comprising multiple copies of a sequence complementary to the circular template strand.

Another aspect provides high-throughput assay methods comprising: (a) providing a plurality of samples in a multiwell format, each sample comprising a partially double-stranded nucleic acid, said nucleic acid comprising a circular template strand and a linear primer strand; (b) adding DNA polymerase and nucleoside triphosphates to each well of the multiwell format; (c) incubating the plurality of samples at conditions suitable to extend the linear primer strand to generate a rolling circle amplification product; (d) hybridizing a detectable nucleic acid probe comprising a donor fluorophore and an acceptor fluorophore to the rolling circle amplification product to form a double-stranded complex, wherein the donor fluorophore and acceptor fluorophore are attached to the detectable nucleic acid probe at positions separated by at least one labile bond; and (e) selectively cleaving the labile bond of detectable probe hybridized to the rolling circle amplification product with a nuclease specific for double-stranded nucleic acids; and (f) detecting a level of fluorescence of the donor fluorophore separated from its acceptor fluorophore in each well of the multiwell format. In some embodiments, the plurality of samples may be derived from tissues or cells cultured in a multiwell format. The multiwell format used for culturing the cells or tissues can be the same or a different multiwell than that used for performing the rolling circle amplification (RCA) and subsequent detection of the RCA product.

In each of these aspects of the invention, the donor fluorophore and acceptor fluorophore typically form a FRET pair comprising a donor fluorophore and an acceptor fluorophore. Exemplary acceptor fluorophores include quenchers, which absorb energy and dissipate it without releasing fluorescence. Quenchers typically dissipate the absorbed energy as heat. The donor fluorophore may be selected from the group consisting of hydroxylcoumarin, methoxycoumarin, Alexa, cascade blue, aminocoumarin, Cy2, FAM, Alexa488, fluorescein isothiocyanate (FITC), Alexa430, Alexa532, HEX, Cy3, TRITC, Alexa546, Alexa555, R-phycoerythrin (PE), Rhodamine Red-X, Tamara, Cy3.5, Rox, Alexa568, Red613, Texas Red, Alexa594, Alexa633, Allophycocyanin, Alexa633, Cy5, Alexa660, Cy5.5, TruRed, Alexa680, Cy7, hexachloro-fluorescein, tetrachloro-fluorescein, TAMRA, ROX, 4-difluoro-5,7-diphenyl-4-bora-3a,4a-diaza-s-indacene-3-propionic acid, 4,4-difluoro-5, p-methoxyphenyl-4-bora-3a,4a-diaza-s-indacene-3-propionic acid, 4,4-difluoro-5-styryl-4-bora-3a,4-adiaza-5-indacene-propionic acid, 6-carboxy-X-rhodamine, N, N, N′,N′-tetramethyl-6-carboxyrhodamine, and eosin, and preferably the donor fluorophore is 6-carboxyfluorescein. The acceptor fluorophore may be selected from the group consisting of DDQ-I, DDQ-II, dabcyl, Eclipse, Iowa Black FQ, Iowa Black RQ, ZEN quencher, BHQ-1, BHQ-2, BHQ-3, QSY7, QSY21, and other quenchers, and preferably the acceptor fluorophore is a ZEN quencher. The detectable probe may comprise one or more copies of the reverse complement of the telomere repeat unit, e.g., one or more copies of the sequence 5′-CCCTAA-3′, such as at least two, three or four copies of said sequence. The detectable probe may comprise the sequence 5′-CCCTAACCCTAA-3′ (SEQ ID NO: 1). The probe may be labeled with a donor fluorophore and acceptor fluorophore FRET pair. As one non-limiting example, the probe having the sequence of SEQ ID NO:1 may be labeled at the 5′ end with a 6-carboxyfluorescein and a ZEN quencher at the first T position (i.e., the fourth base). In exemplary embodiments the donor fluorophore and quencher fluorophore may be between 4 and 10 bases apart, though closer or farther separations may also be utilized. In some embodiments, the detectable nucleic acid probe comprises a telomeric sequence or the reverse complement thereof, which may be of the normal or expected telomeric sequence present in the sample (e.g., TTAGGG in the case of mammalian and other vertebrate cells, having the reverse complement CCCTAA). The acceptor fluorophore is preferably selected such that its absorption spectrum overlaps the donor emission spectrum, thereby promoting effective energy transfer. many quenchers absorb fluorophore emission energies over a wide range of wavelengths, such that a given quencher may absorb fluorescence energy from a variety of fluorophores. For example, the quencher dabcyl may be paired with fluorophores including 6-FAM, TET, JOE, HEX, Cy3, Cy5, TAMRA, ROX, or Texas Red. As another example, the quenchers Iowa Black FQ or Iowa Black may be paired with fluorophores including 6-FAM, Rhodamine Green-X, TET, JOE, HEX, Cy3, Cy5, Rhodamine Red-X, ROX, Texas Red-X, TAMRA, or Texas-613. Properties of exemplary fluorophores are summarized in the following table:

Peak absorbance (Ab) and peak emission (Em) wavelength, stokes
shift (SS), and Extinction Coefficient, (s) for 43 Common Fluorophores^{a}

| Dye | Ab (nM) | Em (nM) | SS (nM) | g [symbol] |
|---|---|---|---|---|
| Acridine | 362 | 462 | 100 | 11.000 |
| AMCA | 353 | 442 | 89 | 19.000 |
| BODIPY FL-Br2 | 531 | 545 | 14 | 75.000 |
| BODIPY 530/550 | 534 | 545 | 10 | 77.000 |
| BODIPY TMR | 544 | 570 | 26 | 56.000 |
| BODIPY 558/568 | 558 | 559 | 11 | 97.000 |
| BODIPY 564/570 | 563 | 569 | 6 | 142.000 |
| BODIPY 576/589 | 575 | 588 | 13 | 83.000 |
| BODIPY 581/591 | 581 | 591 | 10 | 136.000 |
| BODIPY TR | 588 | 616 | 28 | 68.000 |
| BODIPY 630/650* | 625 | 640 | 15 | 101.000 |
| BODIPY 650/665 | 646 | 660 | 14 | 102.000 |
| Cascade Blue | 396 | 410 | 14 | 29.000 |
| Cy2 | 489 | 506 | 17 | 150.000 |
| Cy3* | 552 | 570 | 18 | 150.000 |
| Cy3.5 | 581 | 596 | 15 | 150.000 |
| Cy5* | 643 | 667 | 24 | 250.000 |
| Cy5.5* | 675 | 694 | 19 | 250.000 |
| Cy7 | 743 | 767 | 24 | 250.000 |
| Dabcyl* | 453 | none | 0 | 32.000 |
| Edans | 335 | 493 | 158 | 5.900 |
| Eosin | 521 | 544 | 23 | 95.000 |
| Erythrosin | 529 | 553 | 24 | 90.000 |
| Fuorescein* | 492 | 520 | 28 | 78.000 |
| 6-Fam* | 494 | 518 | 24 | 83.000 |
| TET* | 521 | 536 | 15 | — |
| Joe* | 520 | 548 | 28 | 71.000 |
| HEX* | 535 | 556 | 21 | — |
| LightCycler 640 | 625 | 640 | 15 | 110.000 |
| LightCycler 705 | 685 | 705 | 20 | — |
| NBD | 465 | 535 | 70 | 22.000 |
| Oregon Green 488 | 492 | 517 | 25 | 88.000 |
| Oregon Green 500 | 499 | 519 | 20 | 78.000 |
| Oregon Green 514 | 506 | 526 | 20 | 85.000 |
| Rhodamine 6G | 524 | 550 | 26 | 102.000 |
| Rhodamine Green* | 504 | 532 | 25 | 78.000 |
| Rhodamine Red* | 574 | 594 | 20 | 129.000 |
| Rhodol Green | 496 | 523 | 27 | 63.000 |
| TAMRA* | 565 | 580 | 15 | 91.000 |
| ROX* | 585 | 605 | 20 | 82.000 |
| Texas Red* | 595 | 615 | 20 | 80.000 |
| NED | 546 | 575 | 29 | — |
| VIC | 538 | 554 | 26 | — |

[symbol]

Routinely Offered by IDT [symbol]
Energy capture efficiency [symbol]
FIGURES are given for and activated NHS-ester with a linker arm. [symbol]
indicates data missing or illegible when filed

Yet another aspect of the invention provides methods for assessing alternative lengthening of telomeres (ALT) activity, said method comprising the steps of: (a) providing a sample comprising a partially double-stranded nucleic acid, said nucleic acid comprising a circular template strand and a linear primer strand; (b) extending the linear primer strand to generate a labeled rolling circle amplification product; and (c) detecting the labeled rolling circle amplification product by (1) cleaving the labeled rolling circle product to produce labeled fragments; (2) hybridizing labeled fragments to a capture probe to form a fragment-probe complex; and (3) adding an antibody that binds specifically to the label; and (4) measuring binding of the antibody to said fragment-probe complex. In one implementation, the step of extending the linear primer strand comprises: (1) providing DNA polymerase, a labeled nucleoside triphosphate, and nucleoside triphosphates (dNTPs); and (2) incubating the sample, DNA polymerase and dNTPs under conditions suitable to extend the linear primer and synthesize a single linear strand comprising multiple complementary copies of the circular template strand. In certain embodiments, the nucleoside triphosphates comprise dATP, dGTP, dTTP and dUTP. In those embodiments wherein the nucleoside triphosphates comprise dUTP, the labeled rolling circle product can be cleaved by exposure to uracil-DNA glycosylase (UDG).

In certain embodiments of this aspect, the labeled nucleoside triphosphate may be digoxigenin-11-dUTP. Alternatively, the nucleoside triphosphate can be modified by attachment of biotin, dinitrophenyl, fluorescein, fucose or Texas Red as a label. In some embodiments, the step of detecting the labeled rolling circle amplification product comprises binding the capture probe to a solid support prior to measuring binding of the antibody to the fragment-probe complex. In one such implementation, the capture probe is a biotin-tagged polynucleotide comprising a sequence capable of hybridizing to the rolling circle product or fragments thereof and the solid support is coated with streptavidin.

In some embodiments of any of the above aspects, the partially double-stranded nucleic acid is a telomeric circle, more preferably a C-circle or G-circle, and the presence of fluorescence by the donor fluorophore is indicative of ALT activity. In other embodiments, the partially double-stranded nucleic acid is a T-circle. In some embodiments, the partially double-stranded nucleic acid is a telomeric circle comprising repeats of the sequence (CCCTAA) on the circular template strand and the sequence (TTAGGG)ₙ on the linear primer strand. In other embodiments, the partially double-stranded nucleic acid is a telomeric circle comprising repeats of the sequence (TTAGGG) on the circular template strand and the sequence (CCCTAA)ₙ on the linear primer strand

In certain embodiments of any aspect of the invention, extending the linear primer strand comprises (1) providing DNA polymerase and nucleoside triphosphates (dNTPs); and (2) incubating the sample, DNA polymerase and dNTPs under conditions suitable to extend the linear primer and synthesize a single linear strand comprising multiple copies complementary to the circular template strand. In some embodiments, the DNA polymerase is φ29 (phi29) polymerase. In a preferred implementation, the nuclease specific for double-stranded nucleic acids is exonuclease III, T7 exonuclease, lambda exonuclease or Exonuclease V (RecBCD).

In certain embodiments of any aspect of the invention, the sample may include DNA obtained from a cell. The cell can be derived from a subject suffering from, suspected of suffering from, or predisposed to, a disease or condition associated with abnormal cellular proliferation. In one implementation, the cell is a cancer cell. Preferably the cancer is selected from a sarcoma, a blastoma, a carcinoma, a mesothelioma or an astrocytoma.

The present invention improves upon existing methods and assays with the specific goal of making the current ALT detection assay compatible with high-throughput methods, thus enabling full automation of the procedure for large-scale experimentation on multiple samples simultaneously. These samples may be derived from cells in culture, tumors, tissues or bodily fluids. These methods find application in academic biomedical research as well as in clinical disease diagnosis, disease status determination, analysis of treatment regime efficacy, and the identification of novel therapeutic agents.

### VI. DETAILED DESCRIPTION OF THE SEVERAL VIEWS OF THE DRAWINGS

FIG. 1 provides CC chemiluminescent Dot Blot assay signal for three ALT[+] cell lines (G292, GM847, U2OS). The linear region of this assay includes approximately 1-32 ng of genomic DNA per reaction.

FIG. 2A-C provides a comparison of radioactive and chemiluminescent C-circle (CC) assays. Chemiluminescent CC assays detected signal from ALT[+] genomic DNA (A). Response of chemiluminescent CC assays in the range of 0-512 ng of genomic DNA (ALT[+] and ALT[−]) per reaction are shown (B), and in the 0-32 ng genomic DNA range (C).

FIG. 3 provides a schematic description of the CC T7 exonuclease-assisted target recycling (TEATR). A probe for the RCA product of C-circle (TTAGGG)ₙ is added. The probe contains a donor fluorophore and an acceptor fluorophore. T7 exonuclease is used to degrade any probe that is bound to CC RCA product (forming a double-stranded section). Fluorescence signal is detected from the donor fluorophore once it is separated from its corresponding acceptor by the activity of the exonuclease. T7 exonuclease targets the 5′ end of the probe.

FIG. 4 provides a schematic description the fluorometric exonuclease-assisted target recycling (EATR) CC assay. After the rolling circle amplification reaction is completed a probe and an exonuclease are added. The probe binds to CC RCA product (TTAGGG)ₙ and it contains a donor fluorophore and an acceptor fluorophore. The exonuclease III degrades probe that is bound to CC RCA product, thus increasing fluorescence signal. Exonuclease III targets the 3′ end of the probe.

FIG. 5 illustrates the exonuclease-assisted target recycling method of RCA product detection using Exonuclease III and T7 exonuclease.

FIG. 6A-D provides a comparison between Dot Blot and EATR C-circle (CC) assays. The chemiluminescent CC Dot Blot assay can detect signal from ALT+ genomic DNA after just 2 hours post RCA in 96 well plate format (A). The EATR CC assay (B) can also detect signal after only 2 hours of RCA reaction and with very similar sensitivity as compared to the CC Dot Blot assay (A) in the 0-32 ng range (B). The 143B ALT-samples show minimal signal enhancement, even after 8 hours of RCA, for both assays.

FIG. 7A-B demonstrates the linear range of chemiluminescent Dot Blot and EATR C-circle assays. The chemiluminescent CC Dot Blot assay can detect signal from ALT[+] genomic DNA in the linear region after 8 hours of RCA up to 32-64 ng per reaction (B). The EATR CC assay can detect linear signal after 8 hours of RCA reaction up to 128 ng of genomic DNA per sample (A). The sensitivities of both assays to genomic DNA are overall very similar, as observed above, in the 1-128 ng range, but the CC EATR is more sensitive than CC Dot Blot above 128 ng of gDNA per reaction.

FIG. 8A-B demonstrates that the fluorometric CC EATR assay can be run on genomic DNA isolated from multiwell plates. The assay can be performed on genomic DNA isolated from various multiwell plate formats (A). Scaling C-circle signal to genomic DNA content increases reproducibility among the various types of samples tested (B). The number in parenthesis refers to the number of cell-seeded wells present in each sample.

FIG. 9 provides a schematic diagram of the CC ELISA assay. Immediately this assay distinguishes itself from the radioactive assay through the use and incorporation of DIG-labeled dUTP and dUTP in the rolling circle amplification step. The sites of dUTP incorporation break down during the heat and alkaline pH steps to yield a product that is short enough to bind to the streptavidin-coated plate. The biotin-labeled CCCTAA probe attaches the rolling circle amplification product to the streptavidin at the bottom of the well. The DIG antibody is conjugated to alkaline phosphatase, which ultimately yields a luminescent signal.

FIG. 10 demonstrates that the CC ELISA assay can detect RCA-derived products from circular DNA templates. The positive controls C96 and DIG-TTAGGG yield signal along with ALT positive gDNA (U2OS). The telomerase-positive cancer cell line 143B had similar signal to the blank sample.

FIG. 11 demonstrates that the CC ELISA (immunoassay) method has similar sensitivity to the CC chemiluminescent assay. ALT positive gDNA can be detected using both the CC ELISA and CC chemiluminescent assays with similar sensitivity. Genomic DNA containing C-circles shows increased signal past 200 ng per reaction only when detected by the ELISA-based CC assay.

FIG. 12A-D shows the kinetic behavior of the fluorescent signal obtained from the CC EATR assay when the reaction was run for a total of 14 hours using ALT-positive U2OS and ALT-negative 143B genomic DNA as inputs. The normalized fluorescent signal increases dramatically up to 4-6 hours of reaction for ALT-positive samples, and then reaches a more stable level (A-B), while signals from ALT-negative samples increase at a much slower rate (C-D), as expected.

FIG. 13A-D compares results from the CC Dot Blot to the CC EATR assay for ALT-positive U2OS (A-B) and ALT-negative 143B genomic DNA samples (C-D). CC EATR was just as sensitive or more sensitive than CC Dot Blot for some regions of the curve for the U2OS samples, while 143B samples show slightly more background signal for CC EATR vs CC Dot Blot, especially for samples with high levels of genomic DNA.

FIG. 14 depicts the detection of experimentally modulated ALT levels using the CC EATR assay. Treatment of cells with hydroxyurea (HU) decreases ALT levels. The CC EATR assay detected significant decreases in the C-circle content of ALT-positive cell lines after HU treatment. This experiment was performed in a high-throughput format in a 96-well plates.

FIG. 15A-D demonstrates the greater sensitivity of Exonuclease III versus T7 Exonuclease (A-B) in performing the CC EATR reaction for detection of C-circle-derived products generated during the rolling circle amplification (RCA) step. It also shows that the CC EATR assay detected signal from a single well of a 96-well plate that has been seeded with as few as 10,000 U2OS but not 143B cells per well (C-D) at different concentrations of Exonuclease III per reaction (2.5 to 10 units).

FIG. 16A-D shows that the CC EATR assay could detect C-circle signal from 96-well plates seeded with as few as 5,000 cells per well by modifying the genomic DNA extraction method to include an initial digestion step using ATL buffer (Qiagen) and proteinase K (PK) (20 microliters Proteinase K stock at 20 mg/mL added to 180 microliters sample in ATL buffer) at 56 C for 10 minutes. This modification by using ATL/PK does not cause higher genomic DNA yield (A), but it does generate higher signal when it was normalized by the ALT-negative control cell line (B-C) and it was similar to what was measured using the CC Dot Blot assay (D).

FIG. 17A-B illustrates the increased signal generated by ALT-positive samples run using the CC EATR assay as compared to the CC Dot Blot assay for blots developed using short or long exposure times. The CC EATR assay generates higher background from samples that do not contain C-circles versus the CC Dot Blot (A) although at the lower range of genomic DNA (B) the amount of background was more similar to what was measured using the CC Dot Blot assay, particularly for the shorter exposure times.

FIG. 18A-D further compares CC EATR versus CC Dot Blot by normalizing the data obtained to the background signal from 143B ALT-negative cell line genomic DNA (A-B) or by normalizing the data to samples without any genomic DNA (C-D). Running the CC EATR for just 8 hours was sufficient to achieve similar sensitivities to CC Dot Blot assay at short exposure times, but not at long exposures (A-B). However, for the ALT-positive data normalized to samples without any genomic DNA, the CC EATR results were comparable even to the long exposure CC Dot Blot assay (C-D).

FIG. 19A-D demonstrates dependence of the CC EATR assay signals on the DNA products created from C-circles present in the samples (A-B). The figure additionally illustrates that the assay can be modified to detect the telomeric DNA content present in the samples independent of C-circles (C-D). Omitting the phi29 DNA polymerase enzyme activity, which binds C-circles and creates long single-stranded G-rich repeat sequences, causes a drastic reduction in the C-circle signal generated from ALT-positive genomic DNA to near ALT-negative sample values (A-B). This was observed when samples were heated to 95° C. or 65° C. to inactive the phi29 enzyme. Since heating at high temperatures close to the boiling point of water should denature genomic DNA and thus expose sequences at the telomeres for CC EATR assay probe binding, the lack of phi29 enzyme in the reaction assures that the signals being detected by the CC EATR assay were originating from telomeric DNA sequences present in chromosomal and extrachromosomal elements (C-D). As expected, the telomeric DNA content of U2OS cells, as measured by the CC EATR assay, was significantly higher than in 143B cells, in agreement with results reported in the literature.

### VII. DETAILED DESCRIPTION

### A. Definitions

The term “ALT” as used herein refers to the process of telomere maintenance known as “Alternative Lengthening of Telomeres” or telomerase-independent telomere maintenance. Thus, in its broadest context, “ALT” refers to a means or mechanism of maintaining the length of telomeres (preventing telomere shortening) that is independent of the activity of telomerase. Often referred to as the “ALT mechanism” or “ALT pathway”, it will be appreciated that the invention is not limited by any one specific mechanism by which ALT may operate in a cell to maintain telomeres. Thus the term “mechanism” is used generally in this context rather than referring to a specific biochemical mechanism or pathway by which an “ALT mechanism” operates. There may indeed be more than one specific pathway by which ALT operates.

A “telomeric sequence,” as the term is used herein, refers to a repeating sequence found in a chromosomal telomere. The sequence is recognized to vary from one species to the next. In humans and other mammals (as well as most other vertebrates), the sequence is known to be made up of repeats of the sequence TTAGGG. A telomeric sequence may include sequence differences from the typical repeat unit which are though to result from errors introduced during DNA replication and/or telomere length extension, whether mediated by telomerase or the ALT patyway.

As used herein the term “telomeric circle” means in its broadest sense an extrachromosomal DNA molecule comprising C-rich and/or G-rich telomeric DNA. Though a variety of structures and topologies may be potentially included, telomeric circles typically contain at least one uninterrupted DNA strand (e.g., a G-rich or C-rich strand) that may serve as a template for rolling circle amplification. In the context of the present disclosure, partially double-stranded telomeric circles comprising C-rich telomeric sequences (for example the telomeric repeat (CCCTAA)ₙ) on the circular strand are referred to as “C-circles” and partially double-stranded telomeric circles comprising G-rich telomeric sequences (for example the telomeric repeat (TTAGGG)ₙ) on the circular strand are referred to as “G-circles”. Double-stranded telomeric circles containing both sequences (5′-(CCCTAA)ₙ and 5′-(TTAGGG)ₙ) are referred to as T-circles. Telomeric C-circles and G-circles can be identified by their ability to self-prime RCA of telomeric C-strand and G-strand templates, respectively. For example, C-circles typically contain an uninterrupted template C-strand and at least one free 3′ end on the G-strand. Conversely, G-circles typically contain an uninterrupted template G-strand and at least one free 3′ end on the C-strand. In either case, the free 3′ end can be used to prime DNA synthesis, and synthesis of the uninterrupted template strand can produce rolling circle amplification.

As used herein the term “primer” refers to an oligonucleotide that binds to a specific region of a single stranded template nucleic acid molecule and initiates nucleic acid synthesis via an enzymatic reaction, extending from the 3′ end of the primer and complementary to the sequence of the template molecule. In certain situations, the primer can be naturally occurring. For example C-circles are self-primed, comprising a primer naturally formed by the ALT mechanism.

As used herein the term “sample” refers to any biological sample that comprises nucleic acid molecules, typically comprising DNA and/or RNA. Samples may be tissues, cells or extracts thereof, or may be purified samples of nucleic acid molecules. Use of the term “sample” does not necessarily imply the presence of sequences to be assayed in accordance with the methods described herein within in the sample.

As used herein, “nucleic acids” include polymeric molecules such as deoxyribonucleic acid (DNA), ribonucleic acid (RNA), peptide nucleic acid (PNA), or any sequence of what are commonly referred to as bases joined by a chemical backbone where the bases have the ability to form base pairs or hybridize with a complementary chemical structure. Suitable non-nucleotidic backbones include, for example, polyamide and polymorpholino backbones. The term “nucleic acids” includes oligonucleotide, nucleotide, or polynucleotide sequences, and fragments or portions thereof. The nucleic acid can be provided in any suitable form, e.g., isolated from natural sources, recombinantly produced, or artificially synthesized, can be single- or double-stranded.

The term “oligonucleotide” refers generally to short chain (e.g., less than about 100 nucleotides in length, and typically about 6 to about 50 nucleotides in length) nucleic acids that can be prepared using techniques presently available in the art such as, for example, solid support nucleic acid synthesis, DNA replication, reverse transcription, restriction digest, run-off transcription, or the like. The exact size of the oligonucleotide will depend upon many factors, which in turn will depend upon the ultimate function or use of the oligonucleotide.

As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. For non-natural bases the base-pairing rules include the formation of hydrogen bonds in a manner similar to the Watson-Crick base pairing rules or by hydrophobic, entropic, or van der Waals forces. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

The term “hybridization” or “hybridize” is used in reference to the pairing of complementary nucleic acids. Hybridization and the strength of hybridization (i.e., the strength of the association between the nucleic acids) is influenced by such factors as the degree of complementarity between the nucleic acids, stringency of the hybridization conditions involved, the melting temperature (Tₘ) of the formed hybrid, and the G:C ratio within the nucleic acids.

As used herein, “label” refers to any atom or molecule which can provide a detectable (preferably quantifiable) signal, and which can be attached to a nucleic acid or protein. Labels can provide signals detectable by such techniques as colorimetric, fluorescent, electrophoretic, electrochemical, spectroscopic, chromatographic, densitometric, or radiographic techniques, and the like. Labels can be molecules that do not themselves produce a detectable signal, but when used in conjunction with another label can produce or quench a detectable signal. For example, a label can be a quencher of a quencher-dye pair. Alternatively, the label can be specifically bound by an antibody, such binding then being directly or indirectly detectable.

“Rolling circle amplification” is based on extension of a primer that has hybridized to a circular template. The primer does not necessarily need to be added exogenously, as in the example of G-circles and C-circles where the sequences suitable to prime rolling circle synthesis may be naturally present. Rolling circle amplification may also proceed from the site of a nick or gap in one strand of a DNA duplex, such that synthesis can proceed from a free 3′ end using the other strand as a template. A polymerase is added that extends the primer sequence. As the circular template has no terminus, the polymerase continually extends the circular template resulting in concatamers of a complement of the template. Rolling-circle amplification is generally described in Baner et al. (1998) Nuc. Acids Res. 26:5073-5078; Barany, F. (1991) Proc. Natl. Acad. Sci. USA 88:189-193; and Lizardi et al. (1998) Nat Genet. 19:225-232, all of which are incorporated by reference in their entirety. In rolling circle amplification, the primer initiates amplification by a polymerase enzyme, such as D29 DNA polymerase (the replicative polymerase from the Bacillus subtilis phage phi29), that has strand displacement ability and which allows the production of long single strands of DNA to be produced. Other DNA polymerases may potentially be utilized. Preferred DNA polymerases for use in rolling circle amplification possess strand displacement ability, such as the BST DNA polymerase large fragment (New England Biolabs).

As used herein, the term “FRET” (“fluorescence resonance energy transfer”; also known as “Forster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually less than 10 nm), excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity is a signal generated. The donor fluorophore and acceptor fluorophore are collectively referred to herein as a “FRET pair”.

### B. Fluorometric Assay

One aspect of the invention provides methods for detecting circular nucleic acid, said method comprising the steps of (a) providing a sample comprising a partially double-stranded nucleic acid, said nucleic acid comprising a circular template strand and a linear primer strand; (b) extending the linear primer strand to generate a rolling circle amplification product; and (c) detecting the rolling circle amplification product, wherein detecting the rolling circle amplification product comprises (1) hybridizing a detectable nucleic acid probe to the rolling circle amplification product to form a double-stranded complex, wherein the detectable nucleic acid probe comprises a donor fluorophore and a acceptor fluorophore attached to the detectable nucleic acid probe at positions separated by at least one labile bond; (2) cleaving the labile bond of detectable probe hybridized to the rolling circle amplification product with a nuclease specific for double-stranded nucleic acids; and (3) detecting fluorescence of the donor fluorophore separated from the acceptor fluorophore. In some embodiments, the step of extending the primer strand comprises: (1) providing DNA polymerase and nucleoside triphosphates (dNTPs); and (2) incubating the sample, DNA polymerase and dNTPs under conditions suitable to extend the linear primer and synthesize a single linear strand comprising multiple copies of a sequence complementary to the circular template strand.

Several DNA structures are associated with telomere management, including C-circles, G-circles, and T-circles. The nomenclature indicates whether the sequence is at least partially single-stranded and comprised of sequences 5′-(CCCTAA)ₙ, referred to as C-circles, or 5′-(TTAGGG)ₙ, referred to as G-circles, or is double-stranded (T-circles) and contains both sequences (5′-(CCCTAA)ₙ and 5′-(TTAGGG)ₙ). In some embodiments, the partially double-stranded nucleic acid is a telomeric circle comprising repeats of the sequence (CCCTAA) on the circular template strand and the sequence (TTAGGG)ₙ on the linear primer strand. In other embodiments, the partially double-stranded nucleic acid is a telomeric circle comprising repeats of the sequence (TTAGGG) on the circular template strand and the sequence (CCCTAA)ₙ on the linear primer strand. The telomeric circles can be obtained from a typical preparation of genomic DNA.

Within the context of the present definition, the term “linear” is to be given its broadest interpretation, meaning ‘not circular’, whilst “circular” means a complete, uninterrupted circular DNA strand. Thus, the linear strand may be of a fragment or segment of DNA of any length that is not a complete or uninterrupted circle, the linear strand comprising a 3′ end hybridized to the circular strand and hence able to be extended by a polymerase in a rolling circle replication/amplification mechanism using the closed circular strand as a template, or alternatively the linear strand comprising a 3′ end that is not hybridized to the circular strand but which is processed by a polymerase or other enzyme complex so as to render it capable of being extended by a polymerase in a rolling circle replication/amplification mechanism using the closed circular strand as a template. For example, the linear strand may be a short fragment with minimum nucleotides sufficient to enable hybridization to the circular strand to generate a short double-stranded region, or may comprise sufficient complementary sequence to the circular strand such that the double-strandedness is almost complete between the circular and the linear strand with the linear strand containing a nick, and all intermediates in between. The linear strand may also have one or more 5′ tails that are not hybridized with the circular strand.

The telomeric sequences in the circular and/or linear strand may be variant or mutated telomeric repeat sequences. The circular and/or linear strand may comprise variant and/or mutated telomeric repeat sequences in addition to canonical telomeric repeat sequences. By “variant” and “mutated” is meant that the telomeric sequences differ from canonical telomeric sequences at one or more nucleotide positions, by either base insertion, deletion or substitution. Each or both of the circular and linear strands may also comprise non-telomeric sequences (such that the strand as a whole may or may not be C-rich or G-rich). The strands typically contain sufficient telomeric sequences (or variant or mutated telomeric sequences with sufficient homology to the genomic telomeric sequences) to enable hybridization to telomeric DNA, typically under physiological conditions. Thus, within the context of the above definition, the circular and linear strands need only comprise telomeric sequences and need not be composed predominantly or solely of telomeric sequences in order to be termed “telomeric”. Also within the context of this definition, the term “partially” encompasses any extent of double-strandedness that is not complete double-strandedness.

In certain embodiments of any aspect of the invention, the sample may include DNA obtained from a cell. In other embodiments, the sample may be partially or completely artificially synthesized.

The cell may be derived from a subject suffering from, suspected of suffering from, or predisposed to, a disease or condition associated with abnormal cellular proliferation. By way of example only, the subject may be suspected of having cancer, may be predisposed or otherwise susceptible thereto, may have one or more high risk factors for developing cancer or may have a cancer predisposition syndrome such as Werner Syndrome, Rothmund-Thomson Syndrome or Li-Fraumeni Syndrome. The method may be employed as part of a screening program for such subjects, or a screening program for a broader population.

The disease or condition may be a cancer. The cancer may be selected from, for example, a sarcoma, a blastoma, a carcinoma, a mesothelioma or an astrocytoma. The sarcoma may be osteosarcoma, malignant fibrous histiocytoma, liposarcoma, synovial sarcoma, fibrosarcoma, chondrosarcoma, rhabdomyosarcoma or leiomyosarcoma. The blastoma may be neuroblastoma. The carcinoma may be a non-small cell lung carcinoma such as lung adenocarcinoma or a breast carcinoma. The mesothelioma may be peritoneal mesothelioma. The astrocytoma may be low-grade astrocytoma, anaplastic astrocytoma, or glioblastoma multiforme

In accordance with the methods described herein the presence and/or amount of partially double-stranded circles may be analyzed from isolated cells. Biological samples derived from subjects may also be used in accordance with the methods. A biological sample for use in accordance with embodiments of the invention may comprise one or more fluid or tissue samples, including, for example, blood, urine, sputum, pleural fluid, peritoneal fluid, bronchial and bronchoalveolar lavage fluid, and tissue sections. The sample may comprise fresh, frozen or otherwise stored biological material. The sample may be obtained by fine needle aspiration biopsy thereby enabling the analysis of multiple sections, for example, from the same tumor or diseased tissue, In some circumstances, the sample may undergo treatment, incubation or culturing after extraction from the subject. Typically, biological samples employed in accordance with the invention include blood. The blood may comprise whole blood, serum or more typically plasma. Thus, embodiments of the invention contemplate the development and implementation of simple, high throughput blood-based tests for ALT activity carried out in accordance with methods described herein.

In some embodiments genomic DNA may be extracted from cells prior to analysis. Alternatively, cells may otherwise be treated to make DNA available for analysis, for example a cell lysate could be used with or without denaturation or digestion of cellular proteins. Analysis may also take place in situ after permeabilization of cells with or without prior fixation and/or crosslinking. Techniques for fixation, crosslinking and permeabilization are well known to those skilled in the art.

By virtue of the circular nature of the template strand, the nucleic acids of the present invention are particularly suitable for amplification via a rolling circle amplification method. extending the linear primer strand comprises (1) providing DNA polymerase and nucleoside triphosphates (dNTPs); and (2) incubating the sample, DNA polymerase and dNTPs under conditions suitable to extend the linear primer and synthesize a single linear strand comprising multiple copies complementary to the circular template strand. In exemplary embodiments, the nuclease specific for double-stranded nucleic acids may include exonuclease III, T7 exonuclease, lambda exonuclease Exonuclease V (RecBCD), or a combination thereof.

Since C-circles and G-circles typically contain a linear segment, thus providing a double-stranded region of the circular DNA, rolling circle amplification advantageously will be conducted without requiring the provision an exogenous primer. Rather, the sequence of the linear segment acts as the primer from which DNA polymerase-mediated strand synthesis is initiated. In those circumstances where a single stranded circular nucleic acid occurs in the absence of a complementary linear segment, an artificially synthesized oligonucleotide can be introduced to the sample as the linear primer strand prior to extending the primer to generate a rolling circle amplification product.

φ29 DNA polymerase is one example of a suitable polymerase for use in accordance with embodiments of the invention. φ29 DNA polymerase is a highly processive polymerase with strand displacement ability that usually produces rolling circle amplification products >70 kb in length. However those skilled in the art will recognize that other polymerases having similar characteristics may also be used and are contemplated herein.

The deoxyribonucleotides (dNTPs) are typically selected from dATP, dGTP, dTTP and dCTP although the exact dNTPs to be employed will depend on the sequence of the circular template strand. For example, where the circular strand consists of repeats of the telomeric sequence CCCTAA, the rolling circle amplification may be conducted using the DNA polymerase in the presence of dATP, dGTP and dTTP only. Said dNTPs may optionally further include dUTP, or optionally include dUTP but not dTTP. Where dUTP is present (either together with dTTP or in the absence of dTTP), product can be cleaved by exposure to uracil-DNA glycosylase (UDG).

The detectable nucleic acid probes may be designed to include a FRET pair so that donor emission is quenched in the absence of target by fluorescence resonance energy transfer (FRET) between two fluorophores. The donor fluorophore, in its excited state, may transfer energy to an acceptor fluorophore when the pair is in close proximity. This transfer is always non-radiative and occurs through dipole-dipole coupling. Any process that sufficiently increases the distance between the fluorophores will decrease FRET efficiency such that the donor fluorophore emission can be detected colorimetrically.

“Donor Fluorophores” are fluorophores or luminescent molecules capable of transferring energy to an acceptor fluorophore, thereby generating a detectable fluorescent signal from the acceptor. Donor fluorophores are generally compounds that absorb in the range of about 300 to 900 nm, for example about 350 to 800 nm. Donor fluorophores have a strong molar absorbance coefficient at the desired excitation wavelength, for example greater than about 10³ M⁻¹ cm⁻¹. Example donor fluorophores include hydroxylcoumarin, methoxycoumarin, Alexa, cascade blue, aminocoumarin, Cy2, FAM, Alexa488, fluorescein isothiocyanate (FITC), Alexa430, Alexa532, HEX, Cy3, TRITC, Alexa546, Alexa555, R-phycoerythrin (PE), Rhodamine Red-X, Tamara, Cy3.5, Rox, Alexa568, Red613, Texas Red, Alexa594, Alexa633, Allophycocyanin, Alexa633, Cy5, Alexa660, Cy5.5, TruRed, Alexa680, Cy7, hexachloro-fluorescein, tetrachloro-fluorescein, TAMRA, ROX, 4-difluoro-5,7-diphenyl-4-bora-3a,4a-diaza-s-indacene-3-propionic acid, 4,4-difluoro-5, p-methoxyphenyl-4-bora-3a,4a-diaza-s-indacene-3-propionic acid, 4,4-difluoro-5-styryl-4-bora-3a,4-adiaza-5-indacene-propionic acid, 6-carboxy-X-rhodamine, N,N,N′,N′-tetramethyl-6-carboxyrhodamine, and eosin.

Acceptor fluorophores are chosen so that their excitation spectra overlap with the emission spectrum of the donor. While some acceptor fluorophores also emit in a visible or detectable spectra, such emission is not required for the present invention. There are non-fluorescent acceptors that will quench a wide range of donors. The quencher can absorb fluorescent energy released by the fluorophore when they are in close proximity, For example, a fluorophore and quencher may be bound to a probe which is cleaved after binding to the amplification product to be detected, such that the fluorophore and quencher can become physically separated, resulting in a detectable increase in fluorescence signal from the fluorophore. Examples of donor/acceptor dye pairs for FRET are Fluorescein/Tetramethylrhodamine, TAEDANS™/Fluorescein, EDANS™/Dabcyl, Fluorescein/Fluorescein, BODIPY™ FL/BODIPY™ FL (Molecular Probes, Eugene, Oreg.), and Fluorescein/QSY7™ Other examples of appropriate donor-acceptor FRET pairs will be known to those skilled in the art. In some embodiments, the acceptor fluorophore comprises a dark quencher, which absorbs excitation energy from the donor fluorophore and dissipates the energy as heat. Suitable acceptor fluorophores include, for example, DDQ-I or DDQ-II (Deep Dark Quenchers available from Eurogentec), Dabcyl, Eclipse (Epoch Biosciences), Iowa Black FQ or Iowa Black RQ or ZEN quencher (Integrated DNA Technologies), BHQ-1 or BHQ-2 or BHQ-3 (Black Hole Quenchers are available from Biosearch Technologies), QSY7 or QSY21 (Molecular Probes, Eugene, Oreg.), and the like. In addition, dyes can also be used as an acceptor fluorophore if they absorb the emitted light of another dye. International Patent Publication No. WO2012033848 A1 by Integrated DNA Technologies (which is hereby incorporated by reference in its entirety) identifies the structure of the ZEN quencher, also referred to therein as “iFQ,” as Formula 3 therein, having the following structure:

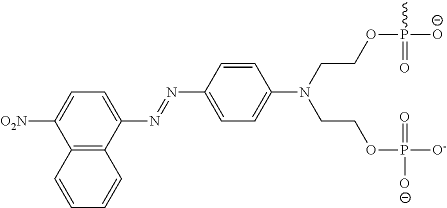 [SMILES: CP(=O)([O-])OCCN(CCOP(=O)([O-])[O-])c1ccc(/N=N/c2ccc([N+](=O)[O-])c3ccccc23)cc1]

Another quencher is dabcyl, having the following molecular structure:

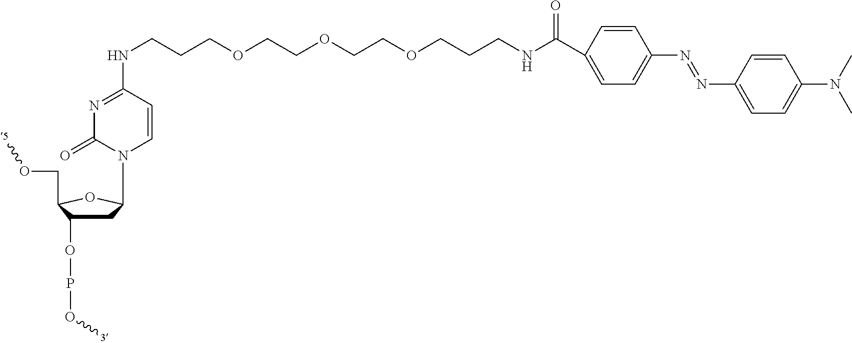

U.S. Pat. Nos. 5,210,015 and 5,487,972, which are each hereby incorporated by reference in their entireties, describe a donor/acceptor fluorescent probe system marketed as TaqMan™ technology by Roche Diagnostics. TAQMAN employs a single stranded oligonucleotide probe that is labeled at the 5′ end with a donor fluorophore and at the 3′ end with an acceptor fluorophore. The DNA polymerase used for amplification in this system contains a 5′->3′ exonuclease activity. The probe binds to one strand of the template at the same time that the primer binds. As the DNA polymerase extends the primer the polymerase will eventually encounter the bound probe. At this time the exonuclease activity of the polymerase will sequentially degrade the probe starting at the 5′ end. As the probe is digested the mononucleotides comprising the probe are released into the reaction buffer, leaving the template stand and nascent amplification product intact.

Assays of this disclosure include both qualitative and quantitative assays. Typical quantitative methods involve measuring a level of fluorescent signal and correlating that level of circular nucleic acid present in the original sample using standard samples containing known amounts of nucleic acid. In a qualitative assay, sufficient fluorescence above or below a threshold level established by samples known to contain or be free of circular nucleic acid establish the assay result. Unless otherwise stated, the act of “measuring” or “determining” in this disclosure refers alternately to qualitative and quantitative determination.

### C. High Throughput Assay

Another aspect provides high-throughput assay methods comprising: (a) providing a plurality of samples in a multiwell format, each sample comprising a partially double-stranded nucleic acid, said nucleic acid comprising a circular template strand and a linear primer strand; (b) adding DNA polymerase and nucleoside triphosphates to each well of the multiwell format; (c) incubating the plurality of samples at conditions suitable to extend the linear primer strand to generate a rolling circle amplification product; (d) hybridizing a detectable nucleic acid probe comprising a donor fluorophore and an acceptor fluorophore to the rolling circle amplification product to form a double-stranded complex, wherein the donor fluorophore and acceptor fluorophore are attached to the detectable nucleic acid probe at positions separated by at least one labile bond; and (e) selectively cleaving the labile bond of detectable probe hybridized to the rolling circle amplification product with a nuclease specific for double-stranded nucleic acids; and (f) detecting a level of fluorescence of the donor fluorophore separated from its acceptor fluorophore in each well of the multiwell format. In some embodiments of this aspect, the plurality of samples is derived from tissues or cells cultured in a multiwell format. The multiwell format used for culturing the cells or tissues can be the same or a different multiwell than that used for performing the rolling circle amplification (RCA) and subsequent detection of the RCA product.

The multiwell format can be any single container incorporating a plurality of assay surfaces (wells), with the number of individual wells ranging from 6 to over 1536. Typical formats are 6-, 12-, 24-, 48-, 96-, 384- and 1536-well plates. Those of skill in the art recognize that there are a wide range of multiwell format types having a standardized footprint, supporting equipment, and measurement systems. Standardized formats allow for ready automation of method steps, including plate handling, pipetting, and spectral excitation, and signal detection. Preferably, the material used to manufacture the multiwell plates is transparent to the excitation and emission wavelengths used by the donor fluorophore of the present method.

### D. Immunoassay

Yet another aspect of the invention provides methods for assessing alternative lengthening of telomeres (ALT) activity, said method comprising the steps of: (a) providing a sample comprising a partially double-stranded nucleic acid, said nucleic acid comprising a circular template strand and a linear primer strand; (b) extending the linear primer strand to generate a labeled rolling circle amplification product; and (c) detecting the labeled rolling circle amplification product by (1) cleaving the labeled rolling circle product to produce labeled fragments; (2) hybridizing labeled fragments to a capture probe to form a fragment-probe complex; and (3) adding an antibody that binds specifically to the label; and (4) measuring binding of the antibody to said fragment-probe complex. In one implementation, the step of extending the linear primer strand comprises: (1) providing DNA polymerase, a labeled nucleoside triphosphate, and nucleoside triphosphates (dNTPs); and (2) incubating the sample, DNA polymerase and dNTPs under conditions suitable to extend the linear primer and synthesize a single linear strand comprising multiple complementary copies of the circular template strand. In certain embodiments, the nucleoside triphosphates comprise dATP, dGTP, dTTP and dUTP. In those embodiments wherein the nucleoside triphosphates comprise dUTP, the labeled rolling circle product can be cleaved by exposure to uracil-DNA glycosylase (UDG).

In certain embodiments of this aspect, the labeled nucleoside triphosphate is digoxigenin-11-dUTP. Alternatively, the nucleoside triphosphate can be modified by attachment of biotin, dinitrophenyl, fluorescein, fucose or Texas Red as a label. In some embodiments, the step of detecting the labeled rolling circle amplification product comprises binding the capture probe to a solid support prior to measuring binding of the antibody to the fragment-probe complex. In one such implementation, the capture probe is a biotin-tagged polynucleotide comprising a sequence capable of hybridizing to the rolling circle product or fragments thereof and the solid support is coated with streptavidin. Examples of solid supports suitable for carrying out the methods disclosed herein include beads, particles, colloids, single surfaces, tubes, multiwell plates, microtiter plates, slides, and electrodes. When the solid support is a particulate material (e.g., beads), it is, in one embodiment, distributed in the wells of multi-well plates to allow for parallel processing of the solid supports.

### VIII. EXAMPLES

A. Chemiluminescence Detection—Dot Blot

CCs were present in the genomic DNA (gDNA) isolated from cells that utilize the ALT pathway to elongate or maintain their telomeres. Genomic DNA was isolated using the DNeasy QIAgen kit according to the manufacturer's instructions and eluted in 10 mM Tris-HCl (pH 7.6) buffer. Genomic DNA was not digested at all by restriction enzymes. Genomic DNA ranging from 0 to 512 ng in 10 μL of sample was transferred into a PCR-grade tube and combined with 10 μL 0.2 mg/ml BSA, 0.1% Tween, 4 mM DTT, 1 mM each dATP, dGTP, dCTP and dTTP, 1× φ29 Buffer and 7.5 U φ29 DNA polymerase (New England Biolabs (NEB)) and incubated at 30° C. for 8 h to allow the rolling circle amplification reaction to take place and then 65° C. for 20 min to heat-inactivate φ29 DNA polymerase.

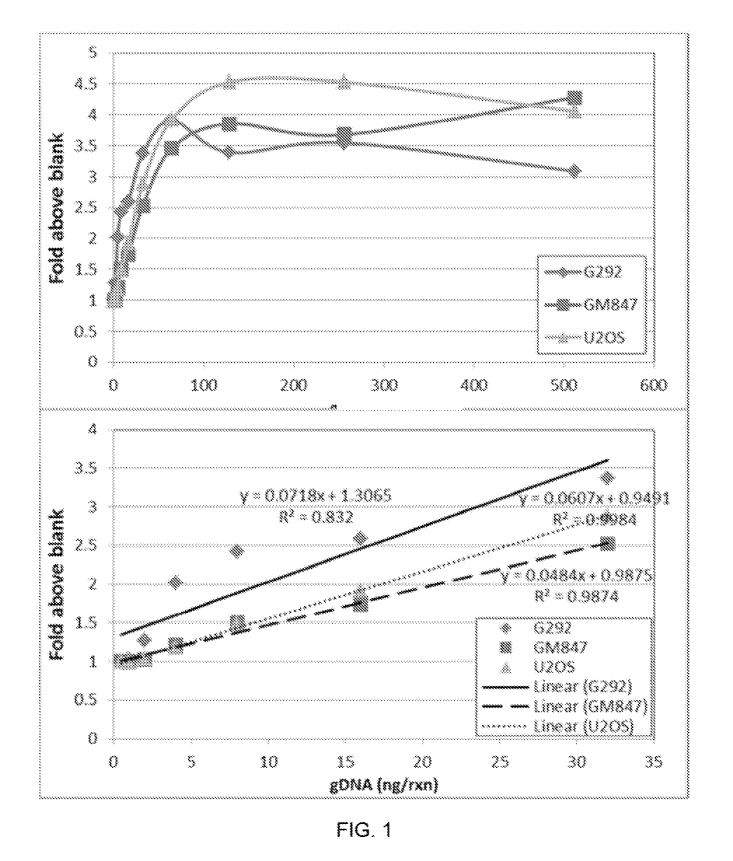

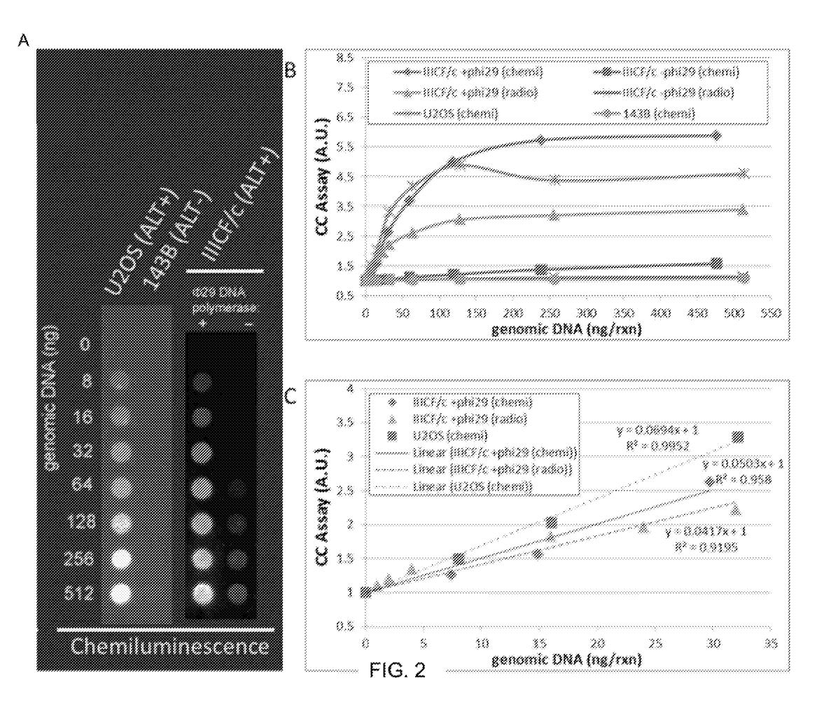

For detection of CC-derived products using a non-radioactive, chemiluminescence-based method, 20 μL of reaction product per sample was diluted to 150 μL with 2×SSC and dot-blotted onto a 2×SSC-soaked Biodyne B nylon membrane (0.45 um, Thermo Scientific). DNA was cross-linked onto the membrane using UV light. The membrane was pre-hybridized in PerfectHyb Plus hybridization buffer (Sigma) for 30 min at 37° C. and then hybridized overnight (12-16 hours) with a 5′ end digoxigenin (DIG)-labeled (CCCTAA)₃ probe in hybridization buffer at 37° C. The membrane was washed and treated as described for the DIG Wash and Block Buffer Set (Roche) using Anti-DIG-AP Fab fragments antibody (Roche). The membrane was developed with CDP-Star reagent (Roche) and chemiluminescence signals were collected using Gene Gnome and analyzed by GeneTools software. Signal for each sample, and for all the comparisons amongst different detection methods disclosed herein, was normalized to the signal obtained from the blank negative controls (0 ng of DNA in the reaction). As shown in FIGS. 1 and 2, the non-radioactive, chemiluminescence-based method of detection was highly sensitive for detection of C-circles in ALT-positive gDNA samples (U2OS, G292, GM847 and IIICF/c cells).

B. Immunoassay—Multi-Well Plates

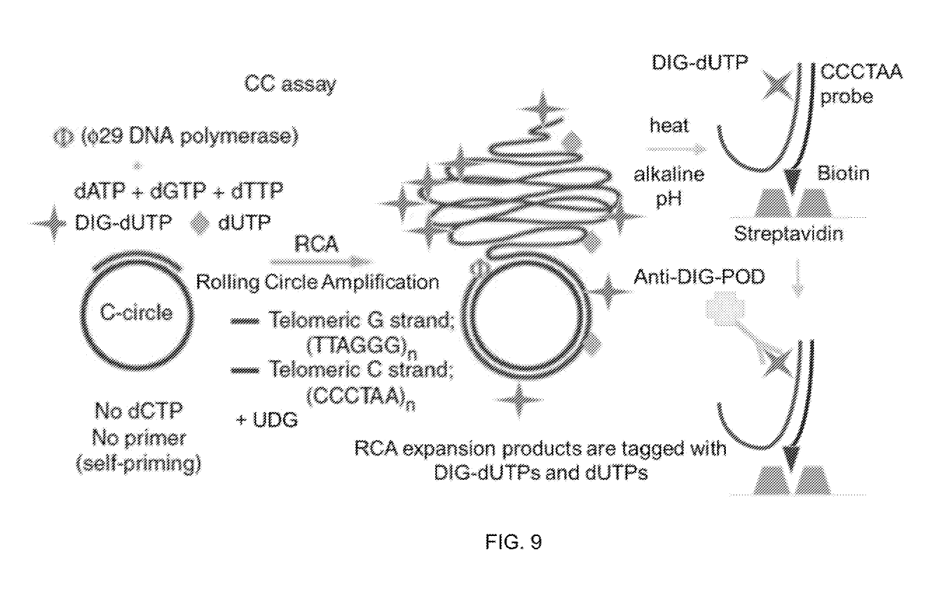

FIG. 9 provides a schematic depiction of an ELISA-based method to quantitatively measure CCs in cells. CCs are present in the genomic DNA of cells that utilize the ALT pathway to elongate or maintain their telomeres. Genomic DNA was isolated using the DNeasy QIAgen kit according to the manufacturer's instructions and eluted in 10 mM Tris-HCl (pH 7.6) buffer. Genomic DNA was not digested at all by restriction enzymes. Genomic DNA ranging from 0 to 512 ng in 10 μL of sample were placed in a PCR-grade tube and combined with 10 μL 0.2 mg/ml BSA, 0.1% Tween, 4 mM DTT, 1 mM each dATP, dGTP, and dCTP, 0.5 mM dTTP, 0.3 mM DIG-labeled dUTP, and 0.2 mM dUTP, 1×φ29 Buffer and 7.5 U (φ29 DNA polymerase (NEB), 0.5 U Uracil-DNA Glycosylase (UDG) and incubated at 30° C. for 8 h to allow the rolling circle amplification (RCA) reaction to take place as well as labeling of the growing CC-derived TTAGGG strands with both DIG-dUTP and dUTP, and then 65° C. for 20 min to heat-inactivate φ29 DNA polymerase and UDG. UDG also digests the dU residues during the RCA reaction.

For the ELISA-based detection of RCA-derived CC products, 20 μL of each sample post-RCA (a single reaction per sample) was run using the PCR ELISA DIG Detection kit and protocol by Roche, according to manufacturer's instructions. Briefly, 20 μL of sample was combined with 20 μL of denaturation solution and incubated for at least 10 min at room temperature. Then 165 μL of hybridization solution was added immediately after the incubation to each sample (205 μL total per sample). Each well of the 96-well microplate from the kit, which was coated with streptavidin to bind biotin, received 200 μL of each sample and the plate was incubated for 3 hours at 37° C. The plate was washed 4 times with 250 μL per well of washing solution and 200 μL of Anti-DIG-POD working solution was added to each well and the plate was incubated for 30 min at 37° C. Plate was washed as above and 200 μL of ABTS substrate solution was added to each well and the plate was incubated for 30 min at 37° C. At the end of this incubation step, the absorbance of each well was immediately read at 405 nm with a reference wavelength at 492 nm. Photometric measurements were made using the Molecular Devices Spectramax Plus 384 microplate reader.

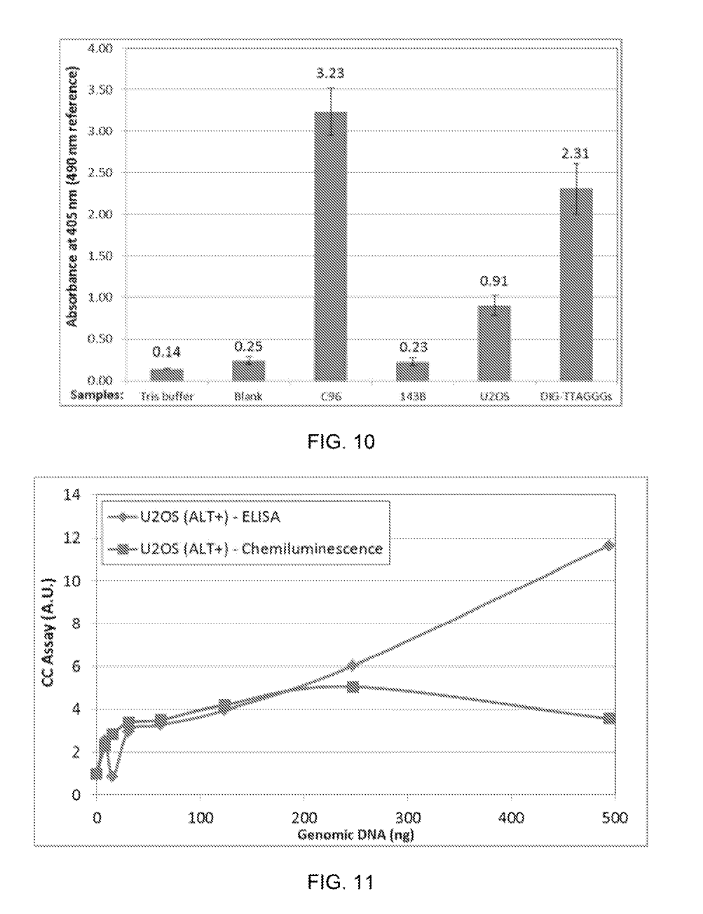

The CC ELISA assay detected signal from the appropriate positive controls, namely the artificial self-priming circular DNA templates (C96) and DIG-labeled TTAGGG oligonucleotides, as well as from the ALT-positive U2OS gDNA sample, but not from the ALT-negative 143B gDNA sample, which was similar in intensity to the blank sample (FIG. 10). As demonstrated in FIG. 11, the CC ELISA assay showed similar sensitivity to ALT-positive genomic DNA as the non-radioactive, chemiluminescence-based version of the CC assay for most of the 0-512 ng/reaction curve. Above 200 ng of gDNA per reaction, the CC ELISA continued to detect greater signal from the samples, while the non-radioactive assay reached complete saturation. Thus, the CC ELISA assay had an overall higher signal-to-noise ratio and greater detection range compared to the non-radioactive CC assay.

C. Fluorescence Detection

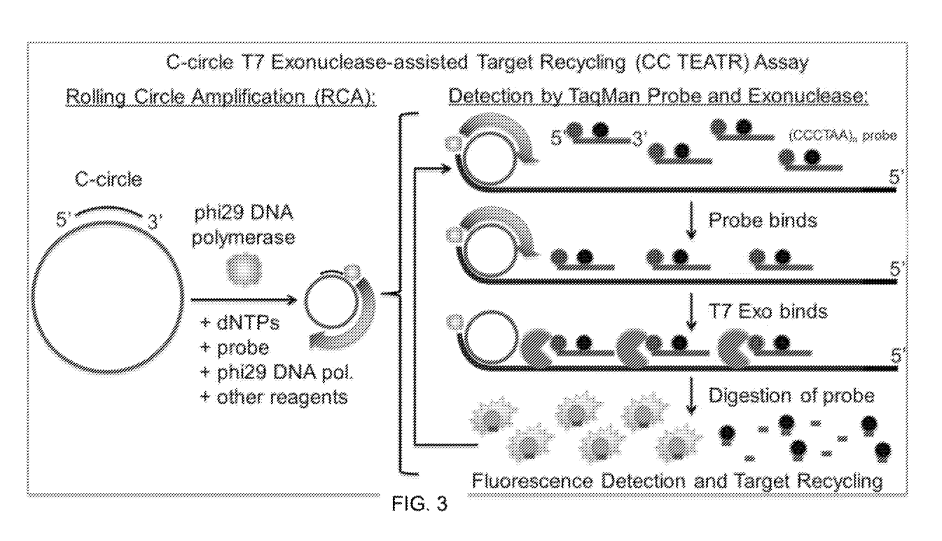

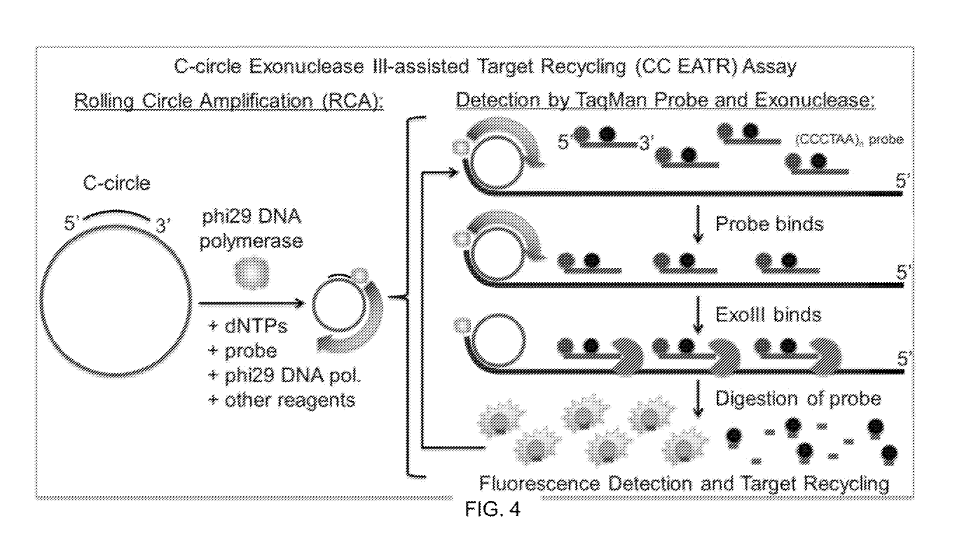

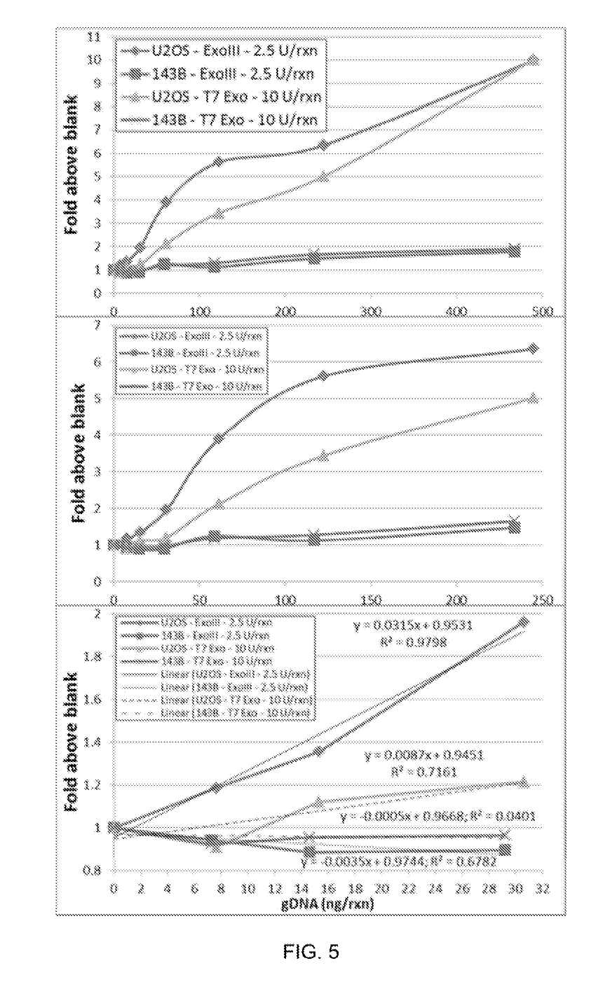

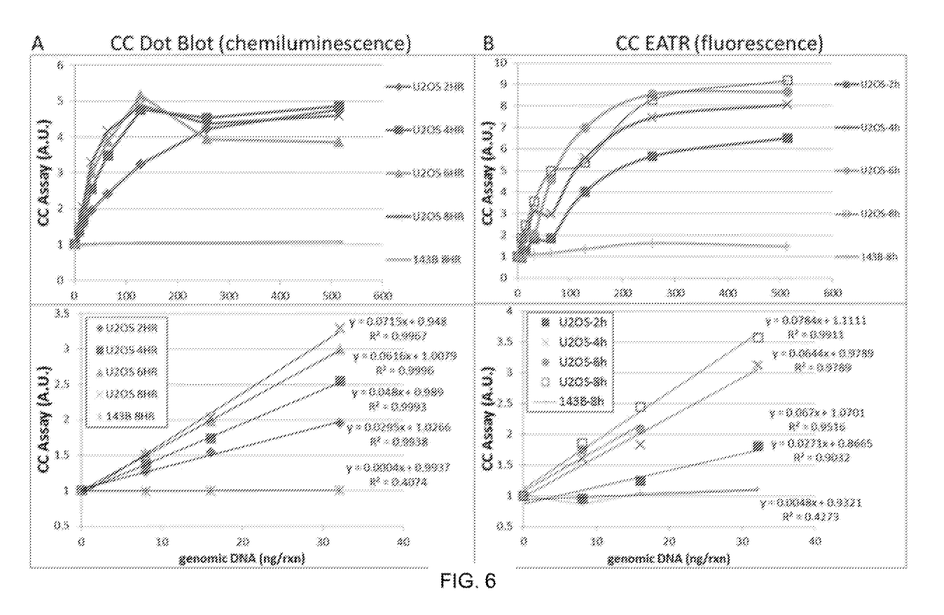

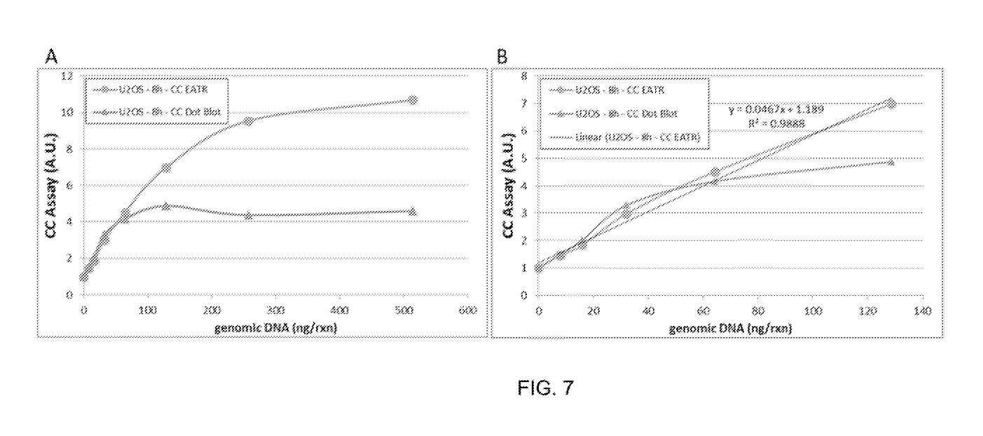

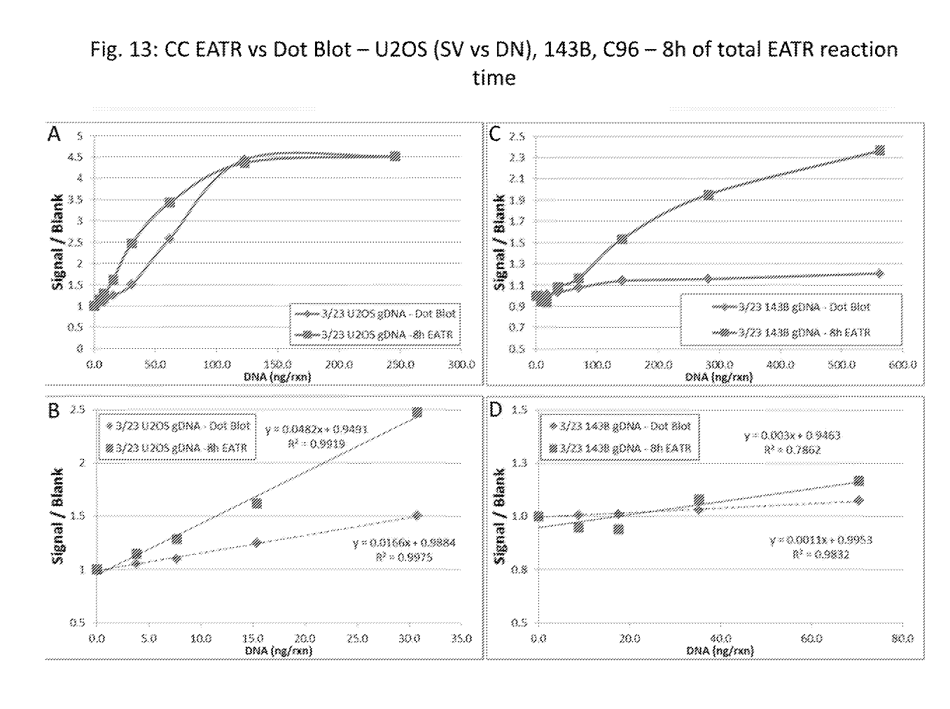

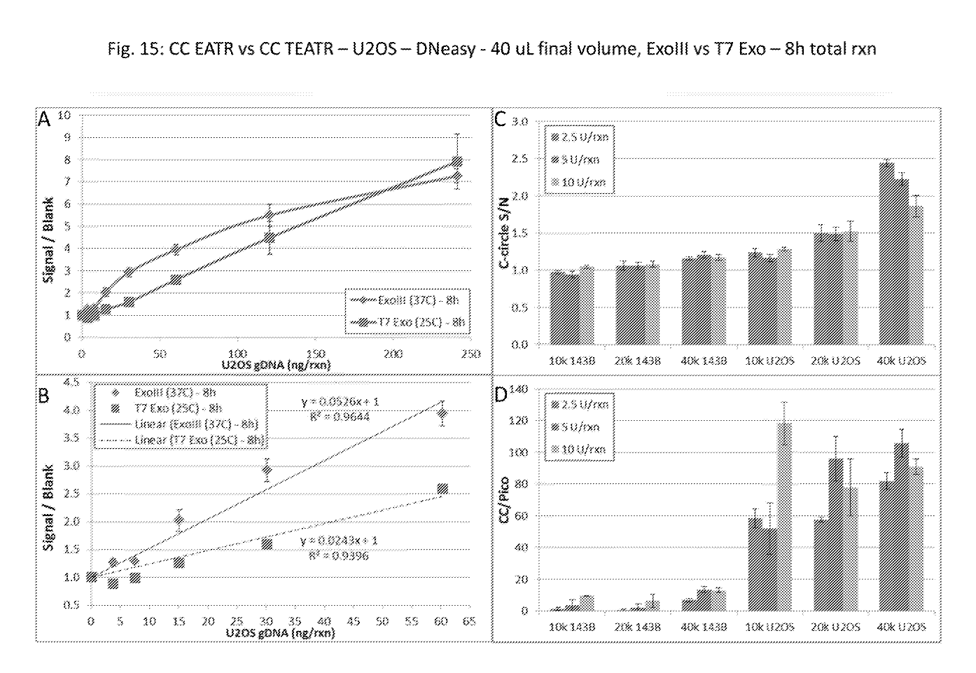

For the fluorescence-based detection of RCA-mediated, CC-derived products (FIGS. 3 and 4, illustrating exonuclease digestion from the 5′ or 3′ terminus, respectively), 20 μL of each sample post-RCA (a single reaction per sample) was combined with 20 μL of 0.1 mg/ml BSA, 3 mM DTT, 0.05% Tween, 10 mM Bis-Tris-Propane-HCl, 10 mM MgCl2 (final pH 7.0 at 25 degrees C.), 2.5 U of Exonuclease III or 10 U of T7 Exonuclease (NEB), and 100 nM of (CCCTAA)₂ probe labeled at the 5′ end with a 6-Carboxyfluorescein (FAM) fluorophore and a ZEN internal quencher (Integrated DNA Technologies) at the first T position. Use of T7 exonuclease to digest the probe is referred to as the CC T7 exonuclease-assisted Target Recycling (TEATR) assay (FIG. 3), but when exonuclease III is used instead, it is referred to as the CC Exonuclease III-assisted Target Recycling (EATR) assay (FIG. 4). Fluorescence signals were collected using Applied Biosystems StepOnePlus Real-Time PCR system configured to acquire data from FAM/FITC filter sets and the reaction was run at 37° C. for 1 hour with signal being collected at 0 min and then every 15 min thereafter. As demonstrated in FIG. 5, both CC TEATR and EATR assays, which use T7 Exonuclease and Exonuclease III, respectively, were capable of detecting signal from ALT-positive gDNA derived from U2OS cells, but not from ALT-negative 143B samples. FIG. 15A-B shows a superior performance by Exonuclease III in detecting C-circle signals from ALT-positive genomic DNA samples. Further, FIG. 6 shows that the fluorometric CC assay exhibited high sensitivity (see also, FIG. 13), with a high signal to noise ratio overall even at 100 ng of gDNA per reaction and above. The background signal observed from ALT-negative samples was similar in both CC EATR and CC Dot Blot assays in the 0 to 80 ng of genomic DNA per reaction range (FIG. 13). Additionally, the fluorometric CC method exhibited a broader linear response overall as compared to the other CC detection methods (FIGS. 2 and 7).

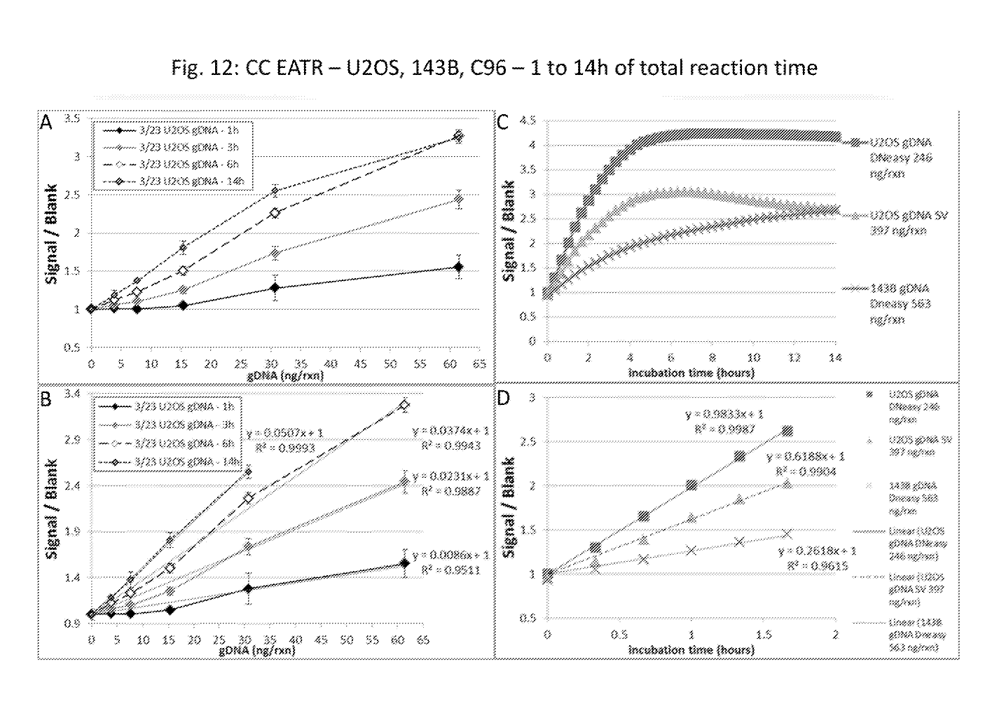

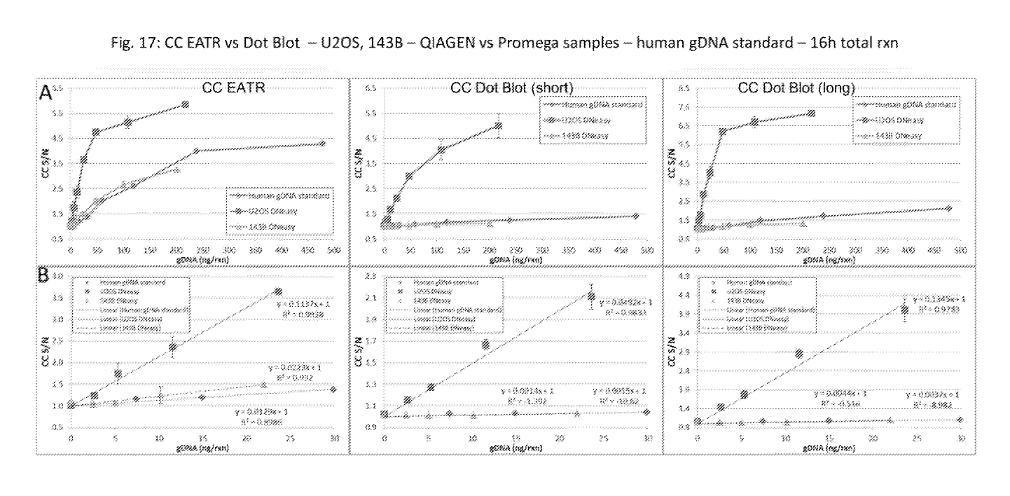

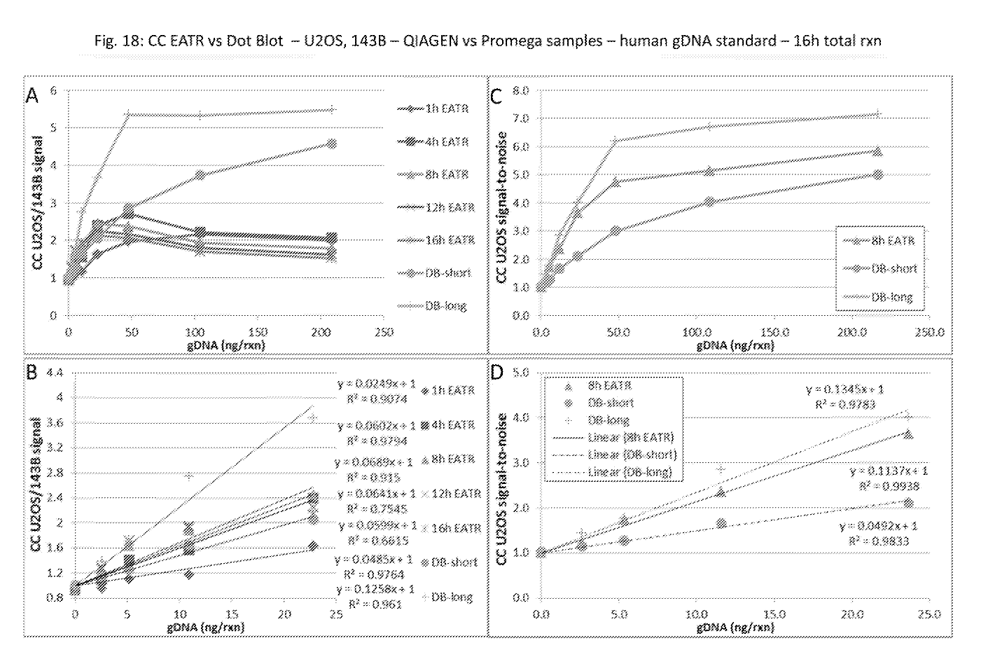

The CC EATR assay also displayed an increase in fluorescence signal that peaked around 4 to 6 hours after the start of the reaction (FIG. 12). The signal from ALT-positive samples increased much more rapidly than from ALT-negative ones over this time-range, resulting in an increased signal-to-background ratio over this time period. The CC EATR assay detected signal from ALT-positive samples with similar sensitivity to the CC Dot Blot assay (FIG. 18), even when the latter was performed at longer exposure times during blot development, but CC EATR also detected higher background signal from ALT-negative samples (FIG. 17).

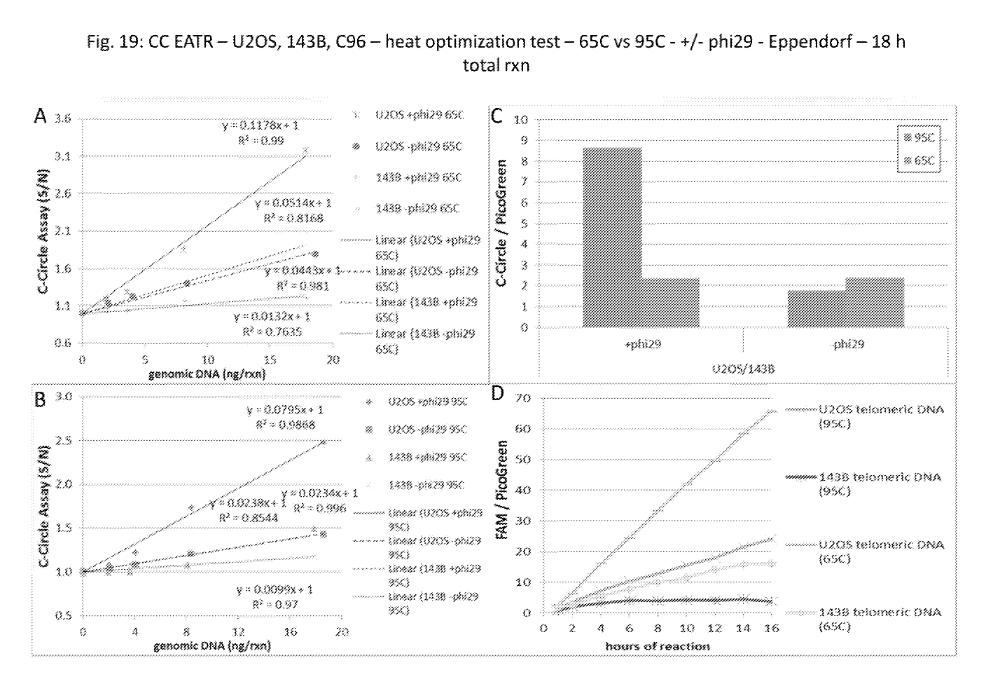

A modified CC EATR assay was also used to detect general telomeric DNA content present in non-ALT cell lines. We adapted the CC EATR assay method by omitting phi29 DNA polymerase from the reaction and by thermally denaturing the genomic DNA samples (by heating to 95° C.). This denaturation step exposed telomeric sequences for binding the CC EATR probe. All other reaction components and conditions remained as described above. FIG. 19C-D shows that the modified CC EATR assay measured total telomeric DNA content from ALT-positive and ALT-negative samples. As expected based on the literature, U2OS cells have a substantially higher amount of telomeric DNA as compared to 143B cells. This method can be used as a functional assessment of ALT activity, since any significant changes to the ALT mechanism are expected to lead to modulations in the total telomeric DNA content of the cells. FIG. 19A-B shows that the signals measured by the CC EATR assay, in its standard format, are overwhelmingly coming from the C-circles present in the samples, indicated by the significantly decreased signal for samples without phi29 enzyme in the RCA reaction.

D. Fluorescence Detection—Multiwell Samples

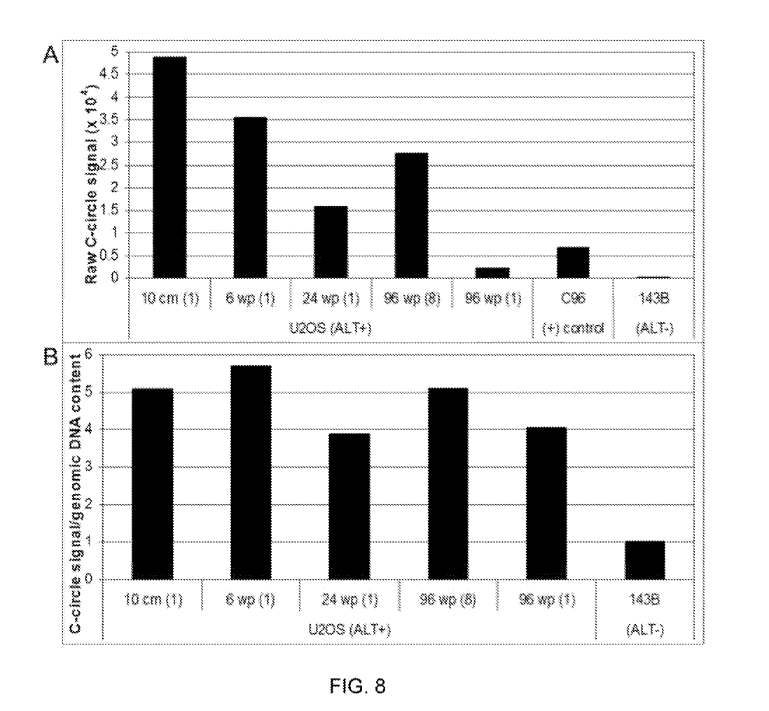

Fluorescence detection-based assays were also be performed using gDNA isolated from samples containing cells cultured and harvested from single wells of 6, 24, and 96 well plates as well as standard 10 cm plates (FIG. 8). This illustrates use of fluorometric CC assays in high-throughput screening formats.

On day 0, passage 50 U2OS cells were seeded into a 10 cm plate, a 6 well plate, a 24 well plate and a 96 well plate at quantities of 5×10⁵, 3×10⁵, 1×10⁴ and 1×10⁴ per well respectively. On day 3, the plates and individual wells were washed twice with 1×PBS before being incubated at 37 C with 1×trypsin for 10 minutes; one condition of eight combined wells of a 96 well plate was included. The trypsin solution was sufficiently mixed, and cell detachment was ensured before transferring solutions to conical tubes. An equal quantity of media was used to inactivate the trypsin before spinning each conical tube at a rate of 300×g. Supernatants were aspirated and pellets were washed in 1×PBS twice. Genomic DNA was isolated using the DNeasy QIAgen kit according to the manufacturer's instructions and eluted in 10 mM Tris-HCl (pH 7.6) buffer. A positive control of C96 produced as per the protocol in Henson et al. 2009 (Nature Biotechnology 27(12):1181-1185) and a negative control of telomerase-positive 143B cell DNA prepared from a 10 cm plate were included. Genomic DNA concentrations of each sample were determined with the Quant-iT™ PicoGreen® dsDNA Assay Kit as per manufacturer's protocol. Genomic DNA was not digested at all by restriction enzymes. 10 μL of genomic DNA sample, for each condition, was transferred into a PCR-grade tube and combined with 10 μL 0.2 mg/ml BSA, 0.1% Tween, 4 mM DTT, 1 mM each dATP, dGTP, dCTP and dTTP, 1×φ29 Buffer and 7.5 U φ29 DNA polymerase (New England Biolabs (NEB)) and incubated at 30° C. for 8 h to allow the rolling circle amplification reaction to take place and then 65° C. for 20 min to heat-inactivate φ29 DNA polymerase. 20 μL of each sample post-RCA (a single reaction per sample) was combined with 20 μL of 0.1 mg/ml BSA, 2 mM DTT, 0.05% Tween, 10 mM Bis-Tris-Propane-HCl, 10 mM MgCl2, 1 mM DTT, 2.5 U of Exonuclease III), and 100 nM of (CCCTAA)₂ probe labeled at the 5′ end with a 6-Carboxyfluorescein (FAM) fluorophore and a ZEN internal quencher (Integrated DNA Technologies) at the first T position (position 4 within the probe sequence). Fluorescence signals were collected using Applied Biosystems StepOnePlus Real-Time PCR system configured to acquire data from FAM/FITC filter sets and the reaction was run at 37° C. for 1 hour with signal being collected at 0 min and then every 15 min thereafter. FIG. 9A represents the raw C-circle signal from the CC EATR reaction run on 10 μL of gDNA isolated from a 10 cm plate; single wells of a 6 well plate, a 24 well plate, a 96 well plate and 8 combined wells of a 96 well plate. C-circle signal can be divided by the genomic DNA concentration, e.g., as determined by PicoGreen, in order to allow comparison of the relative amount of C-circle in samples containing differing gDNA concentrations.

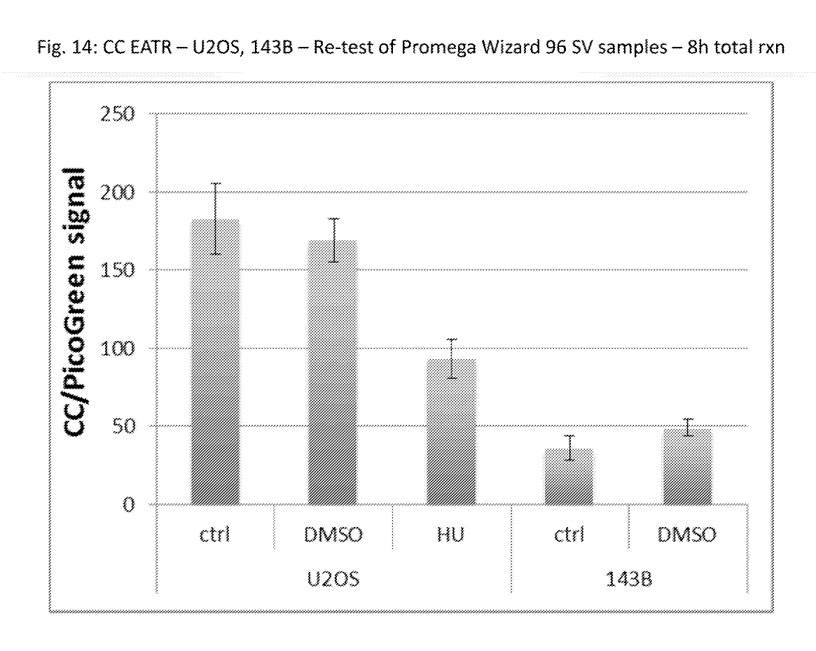

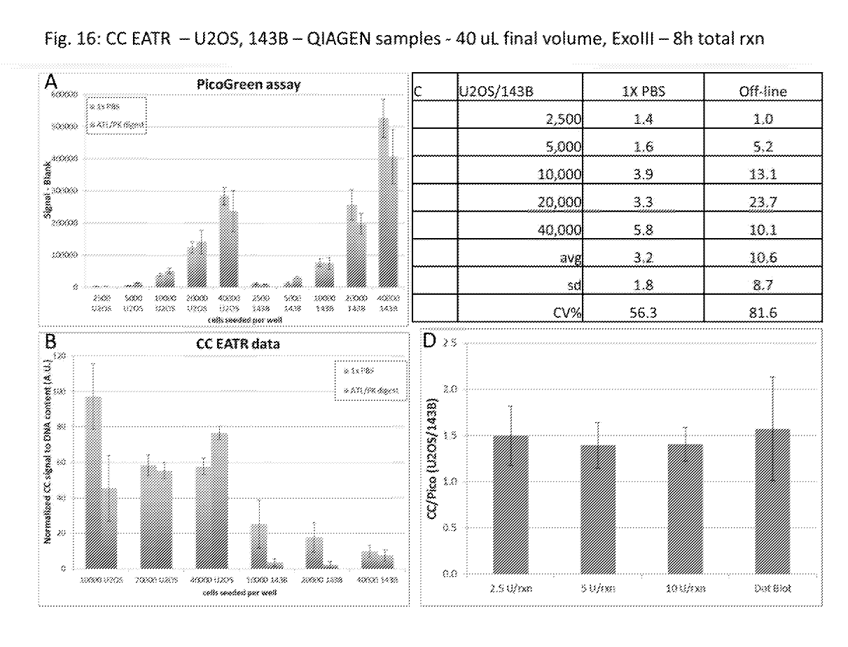

FIG. 14 shows that the high throughput format of the assay using 96-well plates could be used to detect modulation of C-circle content in ALT-positive cell lines treated with an agent that affects ALT. Upon treatment with hydroxyurea (HU), ALT-positive cells have been reported in the literature to exhibit decreased ALT activity. FIGS. 15C-D and FIG. 16 demonstrate that the CC EATR assay was capable of detecting signal from as few as 5,000 ALT-positive cells (which is on a scale readily performed in cell culture laboratories) which were seeded in a single well of a 96-well plate. These data also illustrates the dependence of the assay on Exonuclease III concentration over the range of 2.5 U/reaction to 10 U/reaction (reactions performed in 20 microliter volumes), as well as how the results obtained were compared to measurements performed using the CC Dot Blot assay.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for detection of a circular nucleic acid, said method comprising the steps of:
(a) providing a sample comprising a partially double-stranded nucleic acid, said nucleic acid comprising a circular template strand and a linear primer strand;
(b) extending the linear primer strand to generate a rolling circle amplification product; and
(c) detecting the rolling circle amplification product, wherein detecting the rolling circle amplification product comprises
(1) hybridizing a detectable nucleic acid probe to the rolling circle amplification product to form a double-stranded complex, wherein the detectable nucleic acid probe comprises a donor fluorophore and an acceptor fluorophore attached to the detectable nucleic acid probe at positions separated by at least one labile bond;
(2) cleaving the labile bond of the detectable probe hybridized to the rolling circle amplification product with a nuclease specific for double-stranded nucleic acids; and
(3) detecting fluorescence of the donor fluorophore separated from the acceptor fluorophore.

2. The method of claim 1, wherein the partially double stranded nucleic acid sample comprises a telomeric circle, C-circle, G-circle, or T-circle.

3. (canceled)

4. The method of claim 1, wherein step (a) comprises providing a plurality of samples in a multiwell format, each sample comprising a partially double-stranded nucleic acid, said nucleic acid comprising a circular template strand and a linear primer strand; step (b) comprises adding a DNA polymerase and nucleoside triphosphates to each well of the multiwell format and incubating the plurality of samples at conditions suitable to extend the linear primer strand to generate a rolling circle amplification product.

5. (canceled)

6. (canceled)

7. The method of claim 1, further comprising the step of measuring partially double-stranded nucleic acid in each sample based the level of fluorescence detected in each well of the multiwell format, wherein presence of partially double-stranded nucleic acid is indicative of an active alternative lengthening of telomeres (ALT) mechanism in a sample.

8. (canceled)

9. The method of claim 4, wherein the plurality of samples is derived from tissues or cells cultured or provided in a multiwell format, wherein the multiwell format in which the tissues or cells are cultured or provided is a separate container than the multiwell format in which the samples of step (a) are provided, or wherein the multiwell format in which the tissues or cells are cultured or provided are the same container as the multiwell format in which the samples of step (a) are provided.

10. (canceled)

11. (canceled)

12. (canceled)

13. The method of claim 1, wherein the donor fluorophore is selected from the group consisting of hydroxylcoumarin, methoxycoumarin, Alexa, cascade blue, aminocoumarin, Cy2, FAM, Alexa488, fluorescein isothiocyanate (FITC), Alexa430, Alexa532, HEX, Cy3, TRITC, Alexa546, Alexa555, R-phycoerythrin (PE), Rhodamine Red-X, Tamara, Cy3.5, Rox, Alexa568, Red613, Texas Red, Alexa594, Alexa633, Allophycocyanin, Alexa633, Cy5, Alexa660, Cy5.5, TruRed, Alexa680, Cy7, hexachloro-fluorescein, tetrachloro-fluorescein, TAMRA, ROX, 4-difluoro-5,7-diphenyl-4-bora-3a,4a-diaza-s-indacene-3-propionic acid, 4,4-difluoro-5, p-methoxyphenyl-4-bora-3a,4a-diaza-s-indacene-3-propionic acid, 4,4-difluoro-5-styryl-4-bora-3a,4-adiaza-5-indacene-propionic acid, 6-carboxy-X-rhodamine, N,N,N′,N′-tetramethyl-6-carboxyrhodamine, and eosin and/or wherein the acceptor fluorophore is selected from the group consisting of DDQ-I, DDQ-II, Dabcyl, Eclipse, Iowa Black FQ, Iowa Black RQ, ZEN quencher, BHQ-1, BHQ-2, BHQ-3, QSY7 and QSY21, or wherein the donor fluorophore comprises 6-carboxyfluorescein and the acceptor fluorophore comprises: a ZEN quencher, a napthylene-azo compound, or comprises the structure:

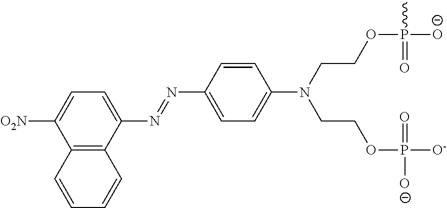 [SMILES: CP(=O)([O-])OCCN(CCOP(=O)([O-])[O-])c1ccc(/N=N/c2ccc([N+](=O)[O-])c3ccccc23)cc1]

14. (canceled)

15. (canceled)

16. The method of claim 1, wherein the detectable nucleic acid probe comprises a telomeric sequence complementary to said rolling circle amplification product, or wherein the detectable nucleic acid probe comprises the sequence CCCTAACCCTAA (SEQ ID NO: 1) or comprises three or more tandem copies of the sequence CCCTAA, and/or wherein said detectable nucleic acid probe comprises said donor fluorophore and said acceptor fluorophore linked to nucleic acid positions within 10 bases, within 20 bases, within 30 bases, within 60 bases, or within between 4 and 10 bases, or within between 4 and 20 bases of each other on said nucleic acid probe, or wherein said donor fluorophore is linked to first base position of said detectable nucleic acid probe and said acceptor fluorophore is linked to the fourth base position of said detectable nucleic acid probe.

17. (canceled)

18. (canceled)

19. (canceled)

20. (canceled)

21. The method of claim 1, wherein the step of extending the linear primer strand comprises:
(1) providing DNA polymerase and nucleoside triphosphates (dNTPs); and
(2) incubating the sample, a DNA polymerase or a DNA polymerase comprising φ29 polymerase, and dNTPs under conditions suitable to extend the linear primer and synthesize a single linear strand comprising multiple copies of a sequence complementary to the circular template strand.

22. (canceled)

23. The method of claim 1, wherein the nuclease specific for double-stranded nucleic acids is selected from the group consisting of exonuclease III, T7 exonuclease, lambda exonuclease, and Exonuclease V; or comprises exonuclease III present in a concentration of at least 0.025 U per microliter, between 0.025 U per microliter and 5 U per microliter, at least 0.125 U per microliter, or between 0.125 U per microliter and 0.5 U per microliter.

24. (canceled)

25. (canceled)

26. (canceled)

27. The method of claim 1, wherein the partially double-stranded nucleic acid comprises repeats of the sequence (CCCTAA) on the circular template strand and comprise the sequence (TTAGGG)ₙ on the linear primer strand, or wherein the partially double-stranded nucleic acid comprises repeats of the sequence (TTAGGG) on the circular template strand and comprise the sequence (CCCTAA)n on the linear primer strand.

28. (canceled)

29. The method of claim 1, wherein (i) the linear primer strand is an artificially synthesized oligonucleotide introduced to the sample prior to extending the linear primer strand to generate a rolling circle amplification product; (ii) said detectable nucleic acid probe is present at a concentration between 5 nM and 4,000 nM or between 50 nM and 200 nM; and/or (iii) the duration of step (c)(2) is at least 1 hour, at least 2 hours, at least 3 hours, at least 4 hours, between 4-6 hours, between 0.5-96 hours, between 1-24 hours, or between 1-12 hours.

30. (canceled)

31. (canceled)

32. (canceled)

33. (canceled)

34. The method of claim 1, wherein the sample is genomic DNA obtained from a cell; genomic DNA obtained from a cell obtained from a subject suffering from, suspected of suffering from, or predisposed to, a disease or condition associated with abnormal cellular proliferation; genomic DNA obtained from a cancer cell; or genomic DNA obtained from a cell of a cancer selected from a sarcoma, a blastoma, a carcinoma, a mesothelioma, an astrocytoma, osteosarcoma, malignant fibrous histiocytoma, liposarcoma, synovial sarcoma, fibrosarcoma, chondrosarcoma, rhabdomyosarcoma, leiomyosarcoma, neuroblastoma, non-small cell lung carcinoma, breast carcinoma, gastric carcinoma, adrenocortical carcinoma, ovarian carcinoma, melanoma, lung adenocarcinoma, peritoneal mesothelioma, or glioblastoma multiforme.

35. (canceled)

36. (canceled)

37. (canceled)

38. The method of claim 34, wherein said cell is treated with an agent that potentially modulates ALT prior to step (a), and said method detects the effects of said agent on the quantity of C-circles in said sample, thereby detecting any increase or decrease in ALT activity that results from said treatment with said agent.

39. The method of claim 1, further comprising comparing the detected level of fluorescence to the level of fluorescence from a standard sample containing a known amount of said circular nucleic acid, thereby determining the quantity of said circular nucleic acid in said sample.

40. A method for assessing alternative lengthening of telomeres (ALT) activity, said method comprising the steps of
(a) providing a sample comprising a partially double-stranded nucleic acid, said nucleic acid comprising a circular template strand and a linear primer strand;
(b) extending the linear primer strand to generate a labeled rolling circle amplification product; and
(c) detecting the labeled rolling circle amplification product by
(1) cleaving the labeled rolling circle product to produce labeled fragments;
(2) hybridizing labeled fragments to a capture probe to form a fragment-probe complex; and
(3) adding an antibody that binds specifically to the label; and
(4) measuring binding of the antibody to said fragment-probe complex.

41. (canceled)

42. (canceled)

43. The method of claim 40, wherein the labeled nucleoside triphosphate is digoxigenin-11-dUTP and optionally the step of cleaving the labeled rolling circle product comprises exposing the labeled rolling circle product to uracil-DNA glycosylase (UDG).

44. (canceled)

45. The method of claim 40, wherein the step of detecting the labeled rolling circle amplification product comprises binding the capture probe to a solid support prior to measuring binding of the antibody to the fragment-probe complex, wherein optionally the capture probe is a biotin-tagged polynucleotide comprising a sequence capable of hybridizing to the rolling circle product or fragments thereof and the solid support is coated with streptavidin.

46. (canceled)

47. The method of claim 1, wherein the method is carried out without addition of a nuclease and/or without addition of a restriction endonuclease to the sample or samples prior to step (a), prior to step (b), and/or prior to step (c), and/or without digestion of genomic DNA in the sample or samples prior to step (a), prior to step (b), and/or prior to step (c), and/or prior to step (a), said sample is digested with a proteinase or with a proteinase comprising proteinase K.

48. (canceled)

49. (canceled)

50. (canceled)

51. (canceled)

52. A detectable nucleic acid probe comprising the sequence CCCTAACCCTAA (SEQ ID NO: 1) linked to a donor fluorophore at position 1 and linked to an acceptor fluorophore at position 4, wherein said donor fluorophore comprises 6-carboxyfluorescein linked to the 5′ end of said detectable nucleic acid probe and said acceptor fluorophore comprises a ZEN quencher, a napthylene-azo compound, or comprises the structure:

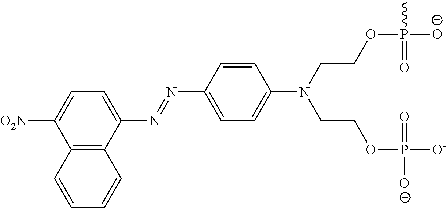 [SMILES: CP(=O)([O-])OCCN(CCOP(=O)([O-])[O-])c1ccc(/N=N/c2ccc([N+](=O)[O-])c3ccccc23)cc1]

53. The detectable nucleic acid probe of claim 52, which is suitable for use in a method for detection of a circular nucleic acid, said method comprising the steps of:
(a) providing a sample comprising a partially double-stranded nucleic acid, said nucleic acid comprising a circular template strand and a linear primer strand;
(b) extending the linear primer strand to generate a rolling circle amplification product and
(c) detecting the rolling circle amplification product, wherein detecting the rolling circle amplification product comprises
(1) hybridizing a detectable nucleic acid probe to the rolling circle amplification product to form a double-stranded complex, wherein the detectable nucleic acid probe comprises a donor fluorophore and an acceptor fluorophore attached to the detectable nucleic acid probe at positions separated by at least one labile bond;
(2) cleaving the labile bond of the detectable probe hybridized to the rolling circle amplification product with a nuclease specific for double-stranded nucleic acids; and
(3) detecting fluorescence of the donor fluorophore separated from the acceptor fluorophore.


SEQUENCE LISTING (1 sequence)

SEQ ID NO: 1 - DNA, 12 bp, Homo sapiens
ccctaaccctaa